(19) United States
(12) Patent Application Publication
(10) Pub. No.: US 2026/0273139 A1
(43) Pub. Date: Sep. 17, 2026

(54) METHOD TO ENGINEER PROTEIN HYDROGELS AND USES THEREOF

(71) Applicant: THE UNIVERSITY OF BRITISH COLUMBIA, Vancouver (CA)

(72) Inventors: Hongbin LI, Vancouver (CA); Linglan FU, Vancouver (CA); Qingyuan BIAN, Vancouver (CA)

(73) Assignee: THE UNIVERSITY OF BRITISH COLUMBIA, Vancouver (CA)

(21) Appl. No.: 19/167,042

(22) PCT Filed: Apr. 12, 2024
(86) PCT No.: PCT/CA2024/050480

§ 371 Date: Sep. 19, 2025

Related U.S. Application Data
(60) Provisional application No. 63/459,105, filed on Apr. 13, 2023.

(51) Int. Cl. A61L 27/52; A61L 27/22; A61L 27/54; C07K 14/78

(52) U.S. Cl. CPC A61L 27/52; A61L 27/227; A61L 27/54; C07K 14/78; A61L 2300/252; A61L 2300/412; A61L 2430/02; A61L 2430/06

(57) ABSTRACT

The present disclosure relates to protein hydrogels, to methods for their preparation and uses thereof, for example in the repair of an osteochondral defect. The protein hydrogels can comprise a crosslinked network of entangled polypeptide chains and at least one amino acid sequence capable as functioning as a biochemical cue.

### CROSS-REFERENCE TO RELATED APPLICATIONS

The present disclosure claims the benefit of priority from co-pending U.S. provisional application No. 63/459,105 filed on Apr. 13, 2023, the contents of which are incorporated herein by reference in their entirety.

### FIELD

The present disclosure relates, for example, to protein hydrogels, to methods for their preparation, and uses thereof, for example in the repair of an osteochondral defect. The protein hydrogels can comprise a crosslinked network of entangled polypeptide chains and at least one amino acid sequence capable as functioning as a biochemical cue.

### BACKGROUND

Load-bearing tissues, such as muscle and cartilage, exhibit mechanical properties that combine high elasticity, high toughness and fast recovery, but display different stiffness, with cartilage being significantly stiffer than muscle (Wainwright et al., 1982; Higuchi, 1996; Linke et al., 1994; Hayes & Mockros, 1971; Temple et al., 2016; Williamson et al., 2003; Kerin et al., 1998; Almarza & Athanasiou, 2004). Muscle achieves its mechanical features via finely-controlled forced unfolding-refolding of protein domains in the giant muscle protein titin. In contrast, articular cartilage achieves its high stiffness and toughness via an entangled supramolecular network made of collagen and proteoglycans. The advance in protein engineering and protein mechanics has made it possible to engineer titin-mimetic elastomeric proteins and use them to engineer soft protein biomaterials (with a Young's modulus <100 kPa) to mimic the passive elastic properties of muscle (Lv et al., 2010; Wu et al., 2018; Khoury et al., 2018). Fast recovery is a hallmark in muscle, where forced-unfolding (i.e. protein unfolding triggered by a stretching force) and refolding of globular protein domains in titin allow for highly effective energy dissipation upon muscle overstretching, and fast recovery upon relaxation (Linke et al., 1994; Rief et al., 1997; Li et al., 2002). This mechanism has been successfully used to engineer soft protein hydrogels whose mechanical properties mimic the passive elastic properties of muscle (Lv et al., 2010; Wu et al., 2018; Khoury et al., 2018; Fang et al., 2013). However, it is challenging to engineer highly stiff and tough protein biomaterials to mimic cartilage (with a modulus in the range of 0.2 to several MPa; Almarza & Athanasiou, 2004), or to develop stiff synthetic extracellular matrices for cartilage stem or progenitor cell differentiation (Jiang & Tuan, 2015), because stiffness and toughness are mutually conflicting.

Load-bearing tissues, ranging from muscle to cartilage, exhibit finely regulated mechanical properties to uniquely suit their biological functions (Wainwright et al., 1982; Gosline et al., 2002). To engineer biomimetics of these biological tissues, protein-based hydrogels have been widely explored (Li, Y. et al., 2020). Protein hydrogels are generally soft, with a Young's modulus smaller than 100 kilopascal (kPa) (Lv et al., 2010; Elvin et al., 2005). Thus, current protein hydrogel technologies have achieved considerable success in achieving mechanical properties that mimic those of softer tissues (Li, Y. et al., 2020; Elvin et al., 2005; McGann et al., 2013), such as muscle (Lv et al., 2010; Wu et al., 2018; Fang et al., 2013).

In comparison, stiffer tissues often have a modulus on the order of megapascal (MPa) and bear tensile as well as compressive loads. For example, articular cartilage is a load-bearing tissue showing a modulus on the order of MPa. It can withstand a load up to a hundred MPa and sustain millions of loading-unloading cycles without much fatigue, and can rapidly recover its shape and mechanical properties after unloading (Wainwright et al., 1982; Kerin et al., 1998; Mccutchen, 1978). Articular cartilage realizes this unique combination of mechanical features by using an entangled network of collagen fiber and proteoglycan (Lu et al., 2008). A double network hydrogel with modulus of 0.2 MPa promoted the regeneration of hyaline cartilage (Yokota et al., 2011; Li, L. et al., 2020) highlighting the importance of improving biomechanical compatibility of hydrogel scaffolds on the regeneration of cartilage. However, due to the mutually conflicting nature of these mechanical properties (high stiffness, high toughness and fast recovery), it is challenging to use current protein hydrogel technology to engineer highly stiff and tough protein hydrogels to mimic the mechanical properties of stiff tissues like cartilage.

Stiff biological tissues, such as cartilage, tendons and ligaments, often integrate seemingly mutually incompatible mechanical properties into themselves (Wainwright et al., 1982). Mimicking such properties using synthetic hydrogels has been challenging, as optimizing one property is often at the expenses of another one. To resolve the incompatibility between high stiffness and high toughness, polymer hydrogels of designed network structures and polymer composite hydrogels have been developed (Gong et al., 2003; Gong, 2010; Xu et al., 2019; Okumura, 2001; Bin Imran et al., 2014; Liu et al., 2017; Wang et al., 2012; Sun et al., 2020), such as double network hydrogels (Gong et al., 2003; Gong, 2010), co-joined network hydrogels (Xu et al., 2019) and slide-ring hydrogels (Okumura, 2001; Bin Imran et al., 2014). Sacrificial bonds/weak secondary networks that can be ruptured are used as the energy dissipation mechanism (Gong, 2010; Sun et al., 2012; Zhao, 2014). Although high stiffness and high toughness have been achieved in some of these hydrogels, slow recovery and mechanical fatigue are often present, due to the irreversible rupture of these sacrificial bonds and/or slow dynamics of weak secondary networks.

Chain entanglement, which arises from the fact that network polymer strands cannot pass through one another in a polymer network, is an important non-covalent mechanism to strengthen polymeric materials (Treloar, 1975). Different from chemical crosslinking, chain entanglement is an entropic effect. Entangled chains are “mobile” in the network and allow mechanical energy to be dissipated in many chains and over long lengths. Thus, chain entanglements will stiffen the polymer network but not make it brittle, a unique feature that has not been fully appreciated. However, in muscle fibers, titin are organized as parallel bundles without chain entanglements (Higuchi, 1996; Linke et al., 1994). In the engineered soft protein hydrogels constructed from elastomeric proteins made of tandem repeats of globular domains, no chain entanglement is present either, due to the short contour length of such elastomeric proteins, about 10 to 40 nm in length (Lv et al., 2010; Fang et al., 2013).

It has been demonstrated that mechanical properties of matrices, such as stiffness, are important physical cues that regulate cell differentiation, tissue development and regeneration, and disease (Engler et al., 2006; Discher et al., 2005). Engineering biomechanically compatible microenvironments has become increasingly important in tissue engineering, but remains challenging in some applications, such as the repair of osteochondral defect (Huey et al., 2012). Osteochondral defect involves lesions of both articular cartilage and the underlying subchondral bone, and occurs due to a traumatic injury to the knee or disorder of the bone (Hunziker, 2002; Hayami et al., 2004). Cartilage regeneration is a complex process and involves many important factors, including biochemical as well as biomechanical ones (Huey et al., 2012). However, there exists an unmet need in engineering biomechanically compatible scaffolds for the repair of osteochondral defect, as no protein-based scaffolds with mechanical properties close to those of cartilage are available.

### SUMMARY

The present disclosure includes a protein hydrogel comprising a crosslinked network of entangled polypeptide chains, wherein the polypeptide chains comprise at least one amino acid sequence capable of functioning as a biochemical cue for tissue repair.

In an embodiment, the biochemical cue is for cartilage repair, bone repair or combinations thereof. In another embodiment, the biochemical cue promotes stem cell adhesion, migration and/or differentiation.

In an embodiment, the at least one amino acid sequence capable of functioning as a biochemical cue comprises a motif capable of entailing cell adhesion to a protein.

In an embodiment, the polypeptide chains further comprise at least one folded globular domain. In another embodiment, the at least one folded globular domain comprises ferredoxin-like folds. In a further embodiment, the protein hydrogel is derived from a protein comprising an unstructured protein sequence positioned between two folded globular domains. In another embodiment, the unstructured protein sequence comprises one of the at least one amino acid sequences capable of functioning as a biochemical cue.

In an embodiment, the protein hydrogel is derived from a protein comprising at least two different amino acid sequences capable of functioning as a biochemical cue, the first amino acid sequence having at least 90% sequence identity to the amino acid sequence set forth in SEQ ID NO:2 and the second amino acid sequence having at least 90% sequence identity to the amino acid sequence set forth in SEQ ID NO:3. In another embodiment, the first amino acid sequence has the amino acid sequence set forth in SEQ ID NO:2 and the second amino acid sequence has the amino acid sequence set forth in SEQ ID NO:3.

In an embodiment, the protein hydrogel is derived from a protein comprising an amino acid sequence having at least 90% sequence identity to the amino acid sequence set forth in SEQ ID NO:10. In another embodiment, the protein hydrogel is derived from a protein comprising the amino acid sequence set forth in SEQ ID NO:10.

In an embodiment, the crosslinks comprise a disulfide bond between cysteine residues in the protein hydrogel.

In an embodiment, the concentration of the crosslinked network of entangled polypeptide chains in the protein hydrogel is about 20% (w/v).

The present disclosure also includes a method of preparing a protein hydrogel, the method comprising:
denaturing a protein in an aqueous environment to produce an aqueous composition comprising overlapping polypeptide chains;
crosslinking the polypeptide chains to produce a denatured protein hydrogel comprising a crosslinked network of entangled polypeptide chains; and
optionally at least partially renaturing the denatured protein hydrogel to produce a renatured protein hydrogel comprising a crosslinked network of entangled polypeptide chains,
wherein the protein comprises at least one amino acid sequence capable of functioning as a biochemical cue for tissue repair.

In an embodiment, the biochemical cue is for cartilage repair, bone repair or combinations thereof. In another embodiment, the biochemical cue promotes stem cell adhesion, migration and/or differentiation.

In an embodiment, the at least one amino acid sequence capable of functioning as a biochemical cue comprises a motif capable of entailing cell adhesion to a protein.

In an embodiment, the protein further comprises at least one folded globular domain. In another embodiment, the at least one folded globular domain comprises ferredoxin-like folds. In a further embodiment, the protein comprises an unstructured protein sequence positioned between two folded globular domains. In another embodiment, the unstructured protein sequence comprises one of the at least one amino acid sequences capable of functioning as a biochemical cue.

In an embodiment, the protein comprises at least two different amino acid sequences capable of functioning as biochemical cues, the first amino acid sequence having at least 90% sequence identity to the amino acid sequence set forth in SEQ ID NO: 2 and the second amino acid sequence having at least 90% sequence identity to the amino acid sequence set forth in SEQ ID NO:3. In another embodiment, the first amino acid sequence has the amino acid sequence set forth in SEQ ID NO:2 and the second amino acid sequence has the amino acid sequence set forth in SEQ ID NO:3.

In an embodiment, the protein comprises the amino acid sequence set forth in SEQ ID NO: 10 or comprises an amino acid sequence having at least 90% sequence identity to the amino acid sequence set forth in SEQ ID NO:10.

In an embodiment, the denaturing comprises subjecting the protein to a chaotropic agent. In another embodiment, the chaotropic agent comprises guanidinium chloride.

In an embodiment, the concentration of the protein in the aqueous environment is about 20% (w/v).

In an embodiment, the method comprises the renaturing of the denatured protein hydrogel to produce the renatured protein hydrogel. In another embodiment, the renaturing comprises equilibrating the denatured protein hydrogel in phosphate buffered saline.

In an embodiment, the crosslinking is carried out in a mold.

In an embodiment, the crosslinks are prepared by a method compatible with use of the protein hydrogel in a subject. In another embodiment, the crosslinks comprise a disulfide bond between cysteine residues obtained by a method comprising exposing the aqueous composition comprising the overlapping polypeptide chains to a source of oxygen.

The present disclosure also includes a protein hydrogel prepared by a method of preparing a protein hydrogel of the present disclosure.

The present disclosure also includes a use of a protein hydrogel of the present disclosure or prepared by a method of preparing a protein hydrogel of the present disclosure in repairing an osteochondral defect.

The present disclosure also includes a synthetic protein comprising an amino acid sequence having at least 90% sequence identity to the amino acid sequence set forth in SEQ ID NO: 10. The present disclosure also includes a synthetic protein having the amino acid sequence set forth in SEQ ID NO:10. The present disclosure also includes a use of such a synthetic protein in preparing a protein hydrogel. In an embodiment, the use is in a method of preparing a protein hydrogel of the present disclosure.

The present disclosure also includes a method of preparing a protein hydrogel, the method comprising:
denaturing a protein in an aqueous environment to produce an aqueous composition comprising overlapping polypeptide chains;
crosslinking the polypeptide chains to produce a denatured protein hydrogel comprising a crosslinked network of entangled polypeptide chains; and
optionally at least partially renaturing the denatured protein hydrogel to produce a renatured protein hydrogel comprising a crosslinked network of entangled polypeptide chains,
wherein the protein comprises at least one amino acid sequence that promotes the adhesion of the protein to a cell.

In an embodiment, the protein comprises (FL)ₓ, (FL-M23C)ₓ, (NuG2)ₓ, (GB1)ₓ, (GA)ₓ, (Cys-FL)ₓ where x is the number of protein repeat units and x is at least 4, GRG5RG4R, N4RN4RNR or combinations thereof.

In an embodiment, the amino acid sequence that promotes the adhesion of the protein to a cell comprises Fn or RGD. In a further embodiment, the protein comprises at least two amino acid sequences that promote adhesion of the protein to a cell.

The present disclosure also includes a protein hydrogel prepared by a method as described herein. The present disclosure further includes a protein hydrogel prepared by a method as described herein with the composition Fn-(Cys-FL)₄-RGD-(Cys-FL)₄-RGD.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should rather be given the broadest interpretation consistent with the description as a whole.

### BRIEF DESCRIPTION OF THE DRAWINGS

The embodiments of the disclosure will now be described in greater detail with reference to the attached drawings, in which:

FIG. 1 shows representative force-distance curves of FL domain at a pulling speed of 50 nm/s. The unfolding-refolding of FL occurred at about 5 pN, making FL a mechanically labile protein. The inset shows the three-dimensional structure of FL (PDB code: 2KL8). FL is a α/β protein, with a four-strand β sheet packing against two α helices.

FIG. 2 shows viscosity measurements of native and denatured (FL)₈ protein solutions in phosphate buffered saline (PBS) and guanidine hydrochloride (GdHCl) in a plot showing viscosity (Pas) as a function of shear rate (1/s). Denatured (FL)₈ (grey triangles) displays higher viscosity than native (FL)₈ (grey diamonds).

FIG. 3 shows schematics of the NC-(FL)₈ (native crosslinked) hydrogels and their preparation.

FIG. 4 shows physical entanglements enhanced the stiffness of the (FL)₈ hydrogels; stress-strain curves of D-DC (*) and D-NC (**) (FL)₈ hydrogels (200 mg/mL) with an inset that is the zoom view of the stress-strain curve of the D-NC hydrogel; and top panel showing the photographs of both hydrogels after being equilibrated in 7M GdHCl.

FIG. 5 shows fluorescence spectra of acid hydrolyzed 20% N-DC and N-NC (FL)₈ hydrogels prepared from the same weight of lyophilized (FL)₈ proteins. Fluorescence at 410 nm is resulted from the dityrosine fluorescence.

FIG. 6 shows stress-strain curves of N-DC (*) and N-NC (**) (FL)₈ hydrogels (200 mg/mL) in PBS, with an inset that is the zoom view of the stress-strain curve of the N-NC hydrogel; and top panel showing the photographs of both hydrogels which were prepared using the same ring-shaped mold equilibrated in PBS. The N-DC hydrogel is translucent, while N-NC hydrogel is opaque. The N-DC hydrogel ruptured at about 100% strain. The mechanical properties of the N-DC hydrogel do not show obvious changes in buffers containing divalent metal ions, such as 10 mM Ca²⁺ or Mg²⁺.

FIG. 7 shows that DC (FL)₈ hydrogels can be cycled between N-DC and D-DC states reversibly. Error bars correspond to the standard deviation, and the number of events n=3.

FIG. 8 shows photographs of scanning electron microscopy (SEM) imaging of the N-DC (upper) and N-NC (lower) (FL)₈ hydrogels. Both hydrogels showed porous network structures. Scale bar in main images show 50 μm. Scale bar in inset in left image shows 10 μm.

FIG. 9 shows schematics of the chain entangled network structure of D-DC and N-DC (FL)₈ hydrogels and their preparation.

FIG. 10 shows typical tensile stress-strain curves of 20% N-DC (FL-M23C)₈ hydrogels. The mechanical properties of (FL-M23C)₈ hydrogels are similar to those of (FL)₈ hydrogels. The Young's modulus is 0.89±0.10 MPa (n=5). Inset shows an optical photograph of the N-DC (FL-M23C)₈ hydrogel.

FIG. 11 shows photographs of (FL-M23C): N-DC hydrogels; a (FL-M23C)₈ N-DC hydrogel under UV illumination light, wherein the blue (observable in color image) fluorescence was from the dityrosine crosslinking points (left); and a (FL-M23C)₈ N-DC hydrogel under UV-illumination after labeling with IAEDANS (5-((2-((iodoacetyl) amino)ethyl)amino) naphthalene-1-sulfonic acid), wherein the cyan (observable in color image) fluorescence was from the labeling of the exposed cysteine residues, and indicated that some FL domains were unfolded in the hydrogel. Scale bars show 5 mm.

FIG. 12 shows fluorescence spectrum of IAEDANS labeled 20% (FL-M23C)₈ hydrogel. Dotted lines are Gaussian fits to the two fluorescence peaks, one is the dityrosine fluorescence at 410 nm, and the other one is the IAEDANS fluorescence at 490 nm.

FIG. 13 shows mechanical properties of N-DC D-DC (FL)₈ hydrogels in tensile testing: Young's modulus (left axis and columns) and breaking strain (right axis and columns) of N-DC and D-DC (FL)₈ hydrogels. It is evident that the N-DC hydrogel exhibited much higher Young's modulus than the N-NC hydrogel. The data is presented as average±standard deviation (ave.+S.D.). The number of events n=18 for both DC and NC hydrogels.

FIG. 14 shows tensile properties of N-DC (FL)₈ hydrogels at different protein concentrations (10%, 15% and 20%): Young's modulus and breaking strain (upper left); and toughness and swelling ratio (upper left) wherein the number of events was n=4 for 10% and 15% hydrogel, and n=18 for 20% hydrogel; and tensile properties of 20% N-DC hydrogels based on (FL)₄, (FL)₈ and (FL)₁₆: tensile modulus and breaking strain (lower left); and toughness and swelling ratio (lower right), wherein n=4 for (FL)₄ and (FL)₁₆, and n=18 for (FL)₈ hydrogels. 20% N-DC (FL)₄ hydrogels showed similar properties as those of (FL); and (FL)₁₆, suggesting that the length of unfolded (FL)₄ is sufficient for chain entanglements.

FIG. 15 shows stretching-relaxation stress-strain curves of the N-DC (FL)₈ hydrogel. The N-DC (FL)₈ hydrogels can dissipate a large amount of energy. A large hysteresis was present in the stretching and relaxation curves, indicative of large energy dissipation.

FIG. 16 shows toughness (left axis and columns) and swelling ratio (right axis and columns) of N-DC and D-DC (FL)₈ hydrogels. It is evident that the N-DC hydrogel exhibited higher toughness than the N-NC hydrogel. The data is presented as average±standard deviation (ave.±S.D.). The number of events n=18 for both DC and NC hydrogels.

FIG. 17 shows the hysteresis between stretching and relaxation curves can be recovered rapidly: the hydrogel was first stretched to about 60% strain and then relaxed to zero strain, after waiting for a certain time Δt, the hydrogel was subject to the stretching-relaxation cycle again and the hysteresis recovery can be directly observed.

FIG. 18 shows the kinetics of the hysteresis recovery in N-DC (FL)₈ hydrogel: about 70% of the hysteresis can be recovered rapidly within a few seconds, and the remaining 30% hysteresis can be recovered following a double-exponential kinetics, a red line (observable in a color image) is a double exponential fit to the data, with a rate constant k₁ of 0.05±0.02 s⁻¹ and k₂ of (1.7±0.3)×10⁻³ s⁻¹, respectively. Error bars are S.D.

FIG. 19 shows exemplary photographs showing that the N-DC (FL)₈ hydrogel can resist cutting with a sharp scalpel: initial state (top image); cut (middle image); and relax (bottom image). Scale bar in top image shows 5 mm.

FIG. 20 shows compressive stress-strain curves of the N-DC (*) and N-NC (FL)₈ (**) hydrogels. Inset is a zoom view of the stress-strain curves of N-NC hydrogel. The N-DC hydrogel can be compressed to more than 80% strain and sustain a compressive stress of >70 MPa without failure. N-DC (FL)₈ hydrogels displayed superb compressive mechanical properties. A large hysteresis was present between the loading and unloading curves, indicating that a large amount of energy was dissipated.

FIG. 21 shows exemplary photographs of the N-DC (FL)₈ hydrogel in its initial state (left); under compression (center top, center bottom); and after unloading, wherein the hydrogel recovered its shape rapidly (right top, right bottom).

FIG. 22 shows schematics of an exemplary hydrogel network structure during compression-unloading.

FIG. 23 shows stress-strain curves of a N-DC (FL)₈ hydrogel compressed to failure. Inset shows the photographs of the hydrogel right after failure (1st cycle; left) and after three more consecutive compression-unloading cycles (4th cycle; right). Cracks were observed right after the failure. Subsequent compression led to the propagation of the crack.

FIG. 24 shows a consecutive compression-unloading curve of the N-DC hydrogel. The hysteresis grows with the increasing of the strain. The toughness of the hydrogel is about 3.2 MJ/m³. The inset is a zoom view of the stress-strain curves at lower strain.

FIG. 25 shows consecutive compression-unloading cycles show that the hysteresis of the N-DC hydrogel can be recovered rapidly. Inset shows the hysteresis recovery kinetics of the hydrogel. About 65% hysteresis can be recovered right after unloading, and the remaining hysteresis can be recovered following a double exponential kinetics, with k₁ of 0.10±0.02 s⁻¹ and k₂ of (2.0±0.3)×10⁻³ s⁻¹.

FIG. 26 shows consecutive loading-unloading curves of the N-DC (FL)₈ hydrogel at a frequency of about 0.08 Hz. The pulling speed was 20 mm/min. In each cycle, the hydrogel was stretched to 50% strain and subsequently relaxed to zero strain. After 600 cycles, the hydrogel displayed little fatigue, and the stress of the hydrogel at 50% strain retained about 83% of the original stress in the first cycle. All the measurements in FIGS. 19, 20 and 24-26 were carried out using 20% N-DC (FL)₈ hydrogels.

FIG. 27 shows consecutive compression-unloading curves of a N-DC (FL)₈ hydrogel at a frequency of 0.08 Hz (top) and 0.67 Hz (bottom). The loading rate was 20 mm/min (top) and 200 mm/min (bottom), respectively.

FIG. 28 shows typical tensile stress-strain curves of 20% N-DC (FL-M23C)₈ hydrogels crosslinked via disulfide bond by air oxygen oxidation overnight. The mechanical properties of (FL-M23C)₈ hydrogels are similar to those of (FL-M23C)₈ hydrogels prepared via photochemical crosslinking. The Young's modulus is 0. 52±0.06 MPa (n=5).

FIGS. 29-30 show cell viability analysis of mouse osteoprogenitor MC3T3-E1 cells cultured on N-DC Fn-(Cys-FL)₄-RGD-(Cys-FL)₄-RGD (FLRGD) hydrogels. FIG. 29 shows epi-fluorescence images of stained cells on N-DC FLRGD hydrogels (On gel; upper row) and cell-culture dish (Control; lower row). Cells were simultaneously stained with Calcein AM dye (center images) and propidium iodide (PI; left images) for staining live and dead cells, respectively. Right images are a Merge. Scale bar shows 200 μm. FIG. 30 shows cell viability determined by live/dead cell staining on N-DC FLRGD hydrogels and cell-culture dish (Control). Error bar corresponds to the standard deviation.

FIG. 31 shows immunostaining of MC3T3-E1 cells after being cultured for 5 days (upper block of 12 images) and 10 days (lower block of 12 images) in blank (upper row in each block of images), N-DC FLRGD hydrogel (center row in each block of images) and PS culturing dish control groups (lower row in each block of images). The Col I (second column from left in each block of images) and Runx2 (far right column in each block of images) are specific markers for the differentiation in the direction of osteoblast. Scale bars show 200 μm.

FIG. 32 shows expression of osteoblast differentiation-related genes, from top to bottom: Col I, RUNX2, ALP and OPN for MC3T3-E1 cells after cultured for 5 days (left plots) and 10 days (right plots). The data represents the relative mRNA levels of the target genes normalized to the levels of the reference genes and are expressed with the levels in the blank group as 1. The values represent the mean±SEM. Differences between groups were assessed by student's t-test; NS: p>0.05; *: p<0.05; **: p<0.01; ***: p<0.001. Cells were cultured on, from left to right in each plot: uncoated cell culture dish (Blank), N-DC FLGRD hydrogel (Gel), and coated cell culture dish (Control).

FIG. 33 shows photographs of the general view of the cartilage and subchondral defect regions of the three groups (from left to right: blank as control; soft N-NC FLRGD/gelatin hydrogel; and stiff N-DC FLRGD hydrogel) at three time points post implantation (from top to bottom rows: week 4, week 8 and week 12).

FIG. 34 shows total International Cartilage Repair Society (ICRS) score (upper left plot) and detailed scoring content of the ICRS score system at week 12: degree of defect repair (upper right plot); integration to border zone (lower left plot); and macroscopic appearance (lower right plot) for, from left to right in each plot: blank as control; soft N-NC FLRGD/gelatin hydrogel; and stiff N-DC FLRGD hydrogel.

FIG. 35 shows microcomputed tomography (micro CT) analysis shows improved subchondral bone repair in the hydrogel group at 12-weeks post-implantation. Obvious newly born bony tissue can be observed in the stiff N-DC FLRGD hydrogel group (right), and the structure of the regenerated bone was similar to the surrounding tissue. In the blank (left) and soft N-NC FLRGD/gelatin hydrogel group (center), a cavity existed in the defect region.

FIG. 36 shows quantitative micro-CT analysis for the three groups; from left to right in each plot: blank as control; soft N-NC FLRGD/gelatin hydrogel; and stiff N-DC FLRGD hydrogel. The stiff hydrogel group exhibited a higher bone volume/total volume (BV/TV (%); upper left plot) and Trabecular Number (Tb.N (1/mm); upper right plot), indicating that significant osteogenesis occurred at the hydrogel treated region. The blank and soft hydrogel groups had a higher Trabecular Separation (Tb.Sp (mm); lower right plot), indicative of the existence of notable bone resorption. The lower left plot shows a comparison of the results for Trabecular Thickness (Tb.Th) in mm between the three groups.

FIG. 37 shows micro magnetic resonance imaging (MRI) analysis. In the stiff FLRGD hydrogel group (right), a consecutive high-brightness structure in the cartilage defect region can be observed, which is similar to the surrounding cartilage tissue. The tissue below cartilage is low contrast with a relatively complete structure, similar to the surrounding bony tissue. In the blank (left) and soft FLRGD/gelatin hydrogel (center) groups, an apparent defect can be found in the cartilage and subchondral bone. The regenerated tissue in the bony region is high-brightness and a distinct boundary with surrounding tissue can be observed. Six animals were scanned and analyzed for each group. The untreated defect (blank) served as a control group. *: p<0.05; **: p<0.01; ***: p<0.001; N.S.: not significant.

FIGS. 38-39 show that histology analysis shows significantly improved subchondral bone and cartilage repair in the stiff FLRGD hydrogel group at 12-weeks post-implantation. The cellular arrangement and structural integrity in stiff FLRGD hydrogel group was similar to the natural bone and cartilage tissues. The ECM staining in the stiff FLRGD hydrogel treated group was also similar to the natural tissue. In contrast, noticeable ECM loss and irregular structure can be observed in the blank and soft FLRGD/gelatin hydrogel groups. FIG. 38 shows histological analysis of cartilage and subchondral bone regeneration with, from top to bottom rows: haematoxylin & Eosin (H&E), safranin O and toluidine blue staining for, from left to right columns: blank, soft FLRGD/gelatin hydrogel and stiff FLRGD hydrogel. Scale bar shows 100 μm. FIG. 39 shows Total O'Driscoll score (upper left plot), and detailed scoring content of O'Driscoll score system: nature of the predominant tissue (upper middle plot); structure characteristics (upper right plot); adjacent cartilage degeneration (lower left plot); and cellular changes (lower right plot) for, from left to right in each plot: blank, soft FLRGD/gelatin hydrogel and stiff FLRGD hydrogel. *: p <0.05; **: p<0.01; ***: p<0.001; N.S.: not significant.

FIGS. 40-46 show tissue remodeling and regeneration process at different time points. FIG. 40 shows the imaging results (top row: micro CT; and second row from top: micro MRI) and histological staining (middle row: H&E; second row from bottom: safranin O; and bottom row: toluidine blue) at week 4 (left three columns, from left to right: blank, soft FLRGD/gelatin hydrogel and stiff FLRGD hydrogel) and week 8 (right columns, from left to right: blank, soft FLRGD/gelatin hydrogel and stiff FLRGD hydrogel). Scale bars show 100 μm. Arrows indicate the remaining hydrogel implants after 4 weeks of implantation. After 8 weeks of implantation, all hydrogel implants were degraded. FIGS. 41-46 show the quantitative analysis at week 4 (FIGS. 41, 43 and 45) and week 8 (FIGS. 42, 44 and 46), including Micro CT analysis (FIGS. 41 and 42), ICRS scoring system (FIGS. 43 and 44), and O'Driscoll scoring system (FIGS. 45 and 46). FIGS. 41 and 42 show plots of bone volume/total volume (BV/TV (%); upper left plots), Trabecular Number (Tb.N (1/mm); upper right plots), Trabecular Thickness (Tb.Th (mm); lower left plots), and Trabecular Separation (Tb.Sp (mm); lower right plots). FIGS. 43 and 44 show plots of total ICRS score (upper left plots), degree of defect repair (upper right plots), integration to border zone (lower left plots), and macroscopic appearance (lower right plots). FIGS. 45 and 46 show plots of Total O'Driscoll score (upper left plots), and detailed scoring content of O'Driscoll score system: nature of the predominant tissue (upper middle plots); structure characteristics (upper right plots); adjacent cartilage degeneration (lower left plots); and cellular changes (lower right plots). From left to right in each plot of FIGS. 41-46: blank, soft FLRGD/gelatin hydrogel and stiff FLRGD hydrogel. No obvious difference was observed at week 4 among the three groups. However, the subchondral bone and cartilage in stiff FLRGD hydrogel treated group exhibited better regeneration phenomenon at week 8. The statistical difference was observed in trabecular parameters and O'Driscoll scoring system. *: p<0.05; **: p<0.01; ***: p<0.001; N.S.: not significant.

FIGS. 47-49 show the immunological response at different time points evaluated using histology staining. FIG. 47 shows the histological analysis (H&E staining) of major organs (from top to second row from bottom: heart, kidney, liver, lung and spleen) and skin (bottom row) at time points of 48 hours (left three columns, from left to right: blank, gelatin and stiff hydrogel) and 7 days (right columns, from left to right: blank, gelatin (negative control) and stiff FLRGD hydrogel). Scale bars show 100 μm. FIG. 48 shows plots of liver function test results (from left to right: alanine transaminase (ALT), aspartate transaminase (AST), albumin (ALB), creatinine (CREA) and cholesterol (CHO)) at two time points; 48 hours (upper plots) and 7 weeks (lower plots) for, from left to right in each plot: blank, gelatin (negative control) and stiff FLRGD hydrogel. FIG. 49 shows histological analysis (H&E staining) of the major organs (from left to right columns: heart, kidney, liver, lung and spleen) from New Zealand rabbits after implantation of gelatin (middle row) or stiff FLRGD hydrogel (lower row) for 12 weeks in comparison to blank (upper row). Scale bars show 100 μm.

FIG. 50 shows the repairing effect of highly stiff (FL-M23C)₈-Fn hydrogel at three time points (from top to bottom rows: 4 weeks, 8 weeks and 12 weeks). Images in the far-left column show the general view of the cartilage and subchondral defects regions at the three time points post implantation of the highly stiff hydrogel. Irregular tissue remained in the defect region after 12 weeks implantation. Micro MRI analysis (second column from the left) and Micro CT analysis (second column from the right) indicated that the regeneration of cartilage regeneration was scarce, and the remolding of subchondral bone was also limited. Images in the far-right column show histological analysis (H&E staining) of cartilage and subchondral bone regeneration. The hydrogel implants (indicated by the arrows) can be clearly observed even after implantation for 12 weeks. Scale bars show 100 μm.

### DETAILED DESCRIPTION

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process/method steps. As used herein, the word “consisting” and its derivatives, are intended to be close ended terms that specify the presence of stated features, elements, components, groups, integers, and/or steps, and also exclude the presence of other unstated features, elements, components, groups, integers and/or steps. The term “consisting essentially of” and any form thereof, as used herein, is intended to specify the presence of the stated features, elements, components, groups, integers, and/or steps as well as those that do not materially affect the basic and novel characteristic(s) of these features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

The term “suitable” as used herein means that the selection of the particular compound, material and/or conditions would depend on the specific synthetic manipulation to be performed, and/or the identity of the compound(s) to be transformed, but the selection would be well within the skill of a person skilled in the art. All method steps described herein are to be conducted under conditions sufficient to provide the product shown.

The expression “proceed to a sufficient extent” as used herein with reference to the reactions or method steps disclosed herein means that the reactions or method steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 100% of the starting material or substrate is converted to product.

The term “sequence identity” as used herein refers to the percentage of sequence identity between two amino acid sequences. Identity can be determined by comparing each position in aligned sequences. A degree of identity between amino acid or nucleic acid sequences is a function of the number of identical or matching amino acids or nucleic acids at positions shared by the sequences, for example, over a specified region (i.e., % identity=number of identical overlapping positions/total number of positions×100%). In one embodiment, the two sequences are the same length. Optimal alignment of sequences for comparisons of identity may be conducted using a variety of algorithms, as are known in the art, including the Clustal WTM program, available at http://clustalw.genome.ad.jp, the local homology algorithm of Smith and Waterman, 1981, Adv. Appl. Math 2:482, the homology alignment algorithm of Needleman and Wunsch, 1970, J. Mol. Biol. 48:443, the search for similarity method of Pearson and Lipman, 1988, Proc. Natl. Acad. Sci. USA 85:2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm (e.g. BLASTn and BLASTp), described in Altschul et al., 1990, J. Mol. Biol. 215:403-10 (using the published default settings). Software for performing BLAST analysis is available through the National Center for Biotechnology Information (http://www.ncbi.nlm.nih.gov/). For instance, sequence identity between two nucleic acid sequences can be determined using the BLASTn algorithm at the following default settings: expect threshold 10; word size 11; match/mismatch scores 2, -3; gap costs existence 5, extension 2. Sequence identity between two amino acid sequences may be determined using the BLASTp algorithm at the following default settings: expect threshold 10; word size 3; matrix BLOSUM 62; gap costs existence 11, extension 1. In another embodiment, the person skilled in the art can readily and properly align any given sequence and deduce sequence identity/homology by mere visual inspection.

The term “subject” as used herein includes all suitable members of the animal kingdom including mammals. In an embodiment, the subject is a human.

### II. Protein Hydrogels. Methods for their Preparation and Uses Thereof

The present disclosure includes a protein hydrogel comprising a crosslinked network of entangled polypeptide chains, wherein the polypeptide chains comprise at least one amino acid sequence that functions as a biochemical cue for tissue repair.

The crosslinked network of entangled polypeptide chains can be derived from any suitable protein. In an embodiment, the crosslinked network of entangled polypeptide chains is derived from an engineered protein. The term “engineered protein” as used herein refers to a polypeptide that does not occur in nature. For example, in an embodiment, the engineered protein comprises at least one change, such as an addition, deletion and/or substitution relative to a naturally occurring polypeptide, wherein such at least one change is introduced by recombinant DNA techniques. In another embodiment, the engineered protein comprises an amino acid sequence generated by man, an artificial protein, a fusion protein or a chimeric polypeptide. Methods of preparing engineered proteins are well known in the art and the selection of a suitable method or source such as a commercial source for a desired engineered protein can be readily made by the skilled person. It will be appreciated by a person skilled in the art that the protein is capable of producing polypeptide chains of a length suitable for entanglement in the protein hydrogels. In an embodiment, the crosslinked network of entangled polypeptide chains is derived from a protein having molecular weight of greater than about 33 kDa. In another embodiment, the crosslinked network of entangled polypeptide chains is derived from a protein having greater than 300 residues. In an embodiment, the polypeptide chains have a length in an unfolded state of at least about 100 nm or at least about 200 nm.

In an embodiment, the biochemical cue is for cartilage repair, bone repair or combinations thereof. In another embodiment, the cartilage is articular cartilage. In an embodiment, the at least one amino acid sequence is capable of functioning as a biochemical cue in the repair of osteochondral defect. Biochemical cues which may be useful in such a repair may include, for example, suitable portions of certain extracellular matrix proteins (e.g., the third FnIII domain of human extracellular matrix protein tenascin and/or RGD, a 17 amino acid residue long sequence, TVYAVTGRGDSPASSRS (SEQ ID NO:3) and/or similar motifs capable of entailing cell adhesion to a protein), other growth factors (e.g., transforming growth factor-β) and/or peptide hormones. In an embodiment, the biochemical cue promotes cell (e.g., stem cell) adhesion, migration and/or differentiation. In another embodiment, the at least one amino acid sequence capable of functioning as a biochemical cue comprises a motif capable of entailing cell adhesion to a protein. In some embodiments, the protein hydrogel is derived from a protein comprising one amino acid sequence capable of functioning as a biochemical cue. In other embodiments, the protein hydrogel is derived from a protein comprising greater than one e.g., at least two, at least three, at least four, at least five, at least six, at least seven, at least eight, at least nine or at least ten or more amino acid sequences capable of functioning as a biochemical cue, for example, the protein can comprise 2, 3, 4, 5, 6, 7, 8, 9 or 10 amino acid sequences capable of functioning as a biochemical cue. In an embodiment, the protein comprises three amino acid sequences capable of functioning as a biochemical cue. In embodiments wherein the protein hydrogel is derived from a protein comprising at least two amino acid sequences capable of functioning as biochemical cues, the sequences can be the same or different. In an embodiment, the protein hydrogel is derived from a protein comprising at least two different amino acid sequences capable of functioning as biochemical cues. For example, in an embodiment, the protein hydrogel is derived from a protein comprising at least two different amino acid sequences capable of functioning as a biochemical cue, the first amino acid sequence having at least 90%, at least 95% or at least 99% sequence identity to the amino acid sequence set forth in SEQ ID NO:2 and the second amino acid sequence having at least 90%, at least 95% or at least 99% sequence identity to the amino acid sequence set forth in SEQ ID NO:3. In another embodiment, the protein hydrogel is derived from a protein comprising at least two different amino acid sequences capable of functioning as a biochemical cue, the first amino acid sequence having at least 90% sequence identity to the amino acid sequence set forth in SEQ ID NO:2 and the second amino acid sequence having at least 90% sequence identity to the amino acid sequence set forth in SEQ ID NO:3. In a further embodiment, the first amino acid sequence has the amino acid sequence set forth in SEQ ID NO:2 and the second amino acid sequence has the amino acid sequence set forth in SEQ ID NO:3. In an embodiment, the protein comprises two copies of the amino acid sequence set forth in SEQ ID NO:3 or having at least 90%, at least 95% or at least 99% sequence identity to the amino acid sequence set forth in SEQ ID NO:3 and one copy of the amino acid sequence set forth in SEQ ID NO:2 or having at least 90%, at least 95% or at least 99% sequence identity to the amino acid sequence set forth in SEQ ID NO:2.

In an embodiment, the polypeptide chains further comprise at least one folded globular domain. In an embodiment, the at least one folded globular domain comprises ferredoxin-like folds. The term “ferredoxin-like folds” as used herein in reference to a protein refers to a motif comprising a topology of 2 α helices and 4 B strands with a ααββαβ secondary structure such that the two terminal β strands hydrogen-bond to the central two β-strands, forming a four-stranded, antiparallel β-sheet covered on one side by two α-helices.

In an embodiment, the at least one folded globular domain comprises repeating motifs having at least 90% sequence identity to the amino acid sequence set forth in SEQ ID NO: 1. In an embodiment, the at least one folded globular domain has been engineered to comprise a cysteine moiety. For example, in an embodiment, the at least one folded globular domain comprises (Cys-FL)ₓ where x is the number of protein repeat units and x is at least 4. In an embodiment, x is an integer of from 4 to 10. In another embodiment, x is an integer of from 4 to 8. In another embodiment, x is an integer of from 4 to 6. In a further embodiment, x is 4. In an embodiment, the protein hydrogel is derived from a protein comprising an unstructured protein sequence positioned between two folded globular domains. In another embodiment, the unstructured protein sequence comprises one of the at least one amino acid sequences capable of functioning as a biochemical cue.

In an embodiment, the protein hydrogel is derived from a protein comprising an amino acid sequence having at least 90%, at least 95% or at least 99% sequence identity to the amino acid sequence set forth in SEQ ID NO: 10. In another embodiment, the protein hydrogel is derived from a protein comprising an amino acid sequence having at least 90% sequence identity to the amino acid sequence set forth in SEQ ID NO:10. In a further embodiment, the protein hydrogel is derived from a protein comprising the amino acid sequence set forth in SEQ ID NO: 10. In an embodiment, the protein is Fn-(Cys-FL)₄-RGD-(Cys-FL)₄-RGD.

Methods of crosslinking proteins are well known in the art and the protein hydrogels of the present disclosure can comprise any suitable crosslinks, chemical or photochemical. In an embodiment, the crosslinks are prepared by a method compatible with use of the protein hydrogel in a subject. For example, in an embodiment, the crosslinks comprise a disulfide bond between cysteine residues. A person skilled in the art would readily appreciate that such crosslinks can be prepared in embodiments wherein the protein hydrogel is derived from a protein comprising suitable cysteine residues, using a method that comprises exposing the cysteine residues to a suitable source of oxygen such as O_{2(g)}, air, H₂O₂ or combinations thereof. In another embodiment, the method comprises exposing the cysteine residues to oxygen in air.

The present disclosure also includes a method of preparing a protein hydrogel, the method comprising:
denaturing a protein in an aqueous environment to produce an aqueous composition comprising overlapping polypeptide chains;
crosslinking the polypeptide chains to produce a denatured protein hydrogel comprising a crosslinked network of entangled polypeptide chains; and
optionally at least partially renaturing the denatured protein hydrogel to produce a renatured protein hydrogel comprising a crosslinked network of entangled polypeptide chains,
wherein the protein comprises at least one amino acid sequence capable of functioning as a biochemical cue for tissue repair.

The denaturing can comprise any suitable method, the selection of which can be made by a person skilled in the art. For example, it would be appreciated by a person skilled in the art that in the methods of preparing a protein hydrogel of the present disclosure, the method of denaturing the protein unfolds the protein thereby producing polypeptide chains that can overlap in the aqueous composition. In some embodiments of the present disclosure, the method of denaturing is desirably reversible, such that, for example, a denatured protein hydrogel can be at least partially renatured to produce a renatured protein hydrogel.

In an embodiment, the denaturing comprises subjecting the protein to a chaotropic agent. The term “chaotropic agent” as used herein refers to an agent that is capable of disrupting the hydrogen bonding network between water molecules and thereby reduces the stability of the native state of the protein by weakening the hydrophobic effect such that the protein is unfolded to produce polypeptide chains that overlap in the aqueous environment of the methods of preparing a protein hydrogel of the present disclosure. In an embodiment, the aqueous environment comprises the chaotropic agent and the method comprises introducing the protein into the aqueous environment. The chaotropic agent is any suitable chaotropic agent. In an embodiment, the chaotropic agent comprises, consists essentially of or consists of guanidinium chloride. In another embodiment, the chaotropic agent comprises guanidinium chloride. In a further embodiment, the chaotropic agent consists essentially of guanidium chloride. In another embodiment, the chaotropic agent consists of guanidium chloride. The concentration of the chaotropic agent is any suitable concentration. For example, a person skilled in the art would appreciate that at high concentrations of guanidium chloride (e.g. about 6M or greater), proteins typically lose their ordered structure which may, for example, produce polypeptide chains suitable for overlapping in the aqueous environment of the methods of preparing a protein hydrogel of the present disclosure. Accordingly, in an embodiment, the concentration of the guanidium chloride in the aqueous environment is in the range of from about 6M to about 8M. In another embodiment, the concentration of the guanidium chloride in the aqueous environment is about 7M.

The concentration of the protein in the aqueous environment is selected such that the polypeptide chains produced from the denaturation of the protein overlap in the aqueous environment. In an embodiment, the concentration of the protein in the aqueous environment is at least 5% (w/v). In another embodiment, the concentration of the protein in the aqueous environment is at least 15% (w/v). In another embodiment, the concentration of the protein in the aqueous environment is from about 15% (w/v) to about 25% (w/v). In another embodiment, the concentration of the protein in the aqueous environment is about 20% (w/v).

In some embodiments, the method comprises the at least partial renaturing of the denatured protein hydrogel to produce the renatured protein hydrogel. In an embodiment, the method comprises the renaturing of the denatured protein hydrogel to produce the renatured protein hydrogel. The renaturing can comprise any suitable method, the selection of which can be made by a person skilled in the art, and may, for example, depend on the method of denaturing. In an embodiment, the renaturing comprises equilibrating the denatured protein in an aqueous composition comprising sodium chloride (e.g. an approximately physiological concentration of sodium chloride) and optionally having a buffer to maintain the aqueous composition at an approximately physiological pH (e.g. a pH of about 7 or from 7.35 to 7.45 or 7.4). In an embodiment, the aqueous composition comprises phosphate buffered saline (e.g. an aqueous composition comprising 137 mM NaCl, 2.7 mM KCl, 10 mM Na₂HPO₄, and 1.8 mM NaH2PO4), Tris-buffered saline or 4-(2-hydroxyethyl)-1-piperazineethanesulfonic acid (HEPES)-buffered saline. In an embodiment, the aqueous composition comprises phosphate buffered saline. In another embodiment, the renaturing comprises equilibrating the denatured protein hydrogel in phosphate buffered saline. The renaturing is carried out for a time and under conditions for the at least partial renaturing of the denatured protein hydrogel to the renatured protein to proceed to a sufficient extent. For example, in an embodiment, the denatured protein hydrogel is contacted with the phosphate buffered saline for a time of from about 8 hours to about 3 days or about 24 hours at ambient temperature such as a temperature of about 4° C. to about 40° C. or about 25° C.

Methods of crosslinking proteins are well known in the art and the methods of preparing a protein hydrogel of the present disclosure can comprise any suitable method of crosslinking, chemical or photochemical. The term “photochemical crosslinking” as used herein refers to methods comprising light irradiation to activate a photoreactive group involved in a chemical reaction to crosslink the polypeptide chains. While the term “chemical crosslinking” may also include “photochemical crosslinking”, the skilled person will appreciate that in certain embodiments herein, for example, wherein it is referred to as an alternative to “photochemical crosslinking” it refers to non-photochemical crosslinking methods such as cysteine-specific crosslinking methods (i.e, methods comprising the use of thiol-reactive reagents to crosslink the polypeptide chains), lysine-specific crosslinking methods (i.e, methods comprising the use of amine-reactive reagents to crosslink the polypeptide chains) and enzymatic crosslinking methods (i.e, methods comprising the use of an enzyme to crosslink the polypeptide chains). In an embodiment, the crosslinks are prepared by a method compatible with use of the protein hydrogel in a subject. In another embodiment, the crosslinks comprise a disulfide bond between cysteine residues obtained by a method comprising exposing the aqueous composition comprising the overlapping polypeptide chains to a source of oxygen. The source of oxygen can be any suitable source, the selection of which can be made by a person skilled in the art. In an embodiment, the source of oxygen comprises O_{2(g)}, air, H₂O₂ or combinations thereof. In another embodiment, the method comprises exposing the cysteine residues to oxygen in air. In some embodiments, the crosslinking is carried out in a mold. For example, in an embodiment, the aqueous composition comprising overlapping polypeptide chains is introduced into a suitable mold (e.g. a mold comprising plexiglass), and subjected to crosslinking for a time for the crosslinking of the polypeptide chains to produce the denatured protein hydrogel to proceed to a sufficient extent. In an embodiment, the method further comprises removing the denatured protein hydrogel from the mold. The conditions for the crosslinking such as the time and/or the temperature may depend, for example, on the method of crosslinking but can be readily selected by a person skilled in the art.

The protein is any suitable protein. In an embodiment, protein is an engineered protein. It will be appreciated by a person skilled in the art that the protein is capable of producing polypeptide chains of a length suitable for overlapping in the aqueous environment. In an embodiment, the protein has a molecular weight of greater than 33 kDa. In another embodiment, the protein has greater than 300 residues. In an embodiment, the polypeptide chains have a length of at least about 100 nm or at least about 200 nm.

In an embodiment, the biochemical cue is for cartilage repair, bone repair or combinations thereof. In another embodiment, the cartilage is articular cartilage. In an embodiment, the at least one amino acid sequence is capable of functioning as a biochemical cue in the repair of osteochondral defect. Biochemical cues which may be useful in such a repair may include, for example, suitable portions of certain extracellular matrix proteins (e.g., the third FnIII domain of human extracellular matrix protein tenascin and/or RGD, a 17 amino acid residue long sequence, TVYAVTGRGDSPASSRS (SEQ ID NO:3) and/or similar motifs capable of entailing cell adhesion to a protein), other growth factors (e.g., transforming growth factor-β) and/or peptide hormones. In an embodiment, the biochemical cue promotes cell (e.g., stem cell) adhesion, migration and/or differentiation. In another embodiment, the at least one amino acid sequence capable of functioning as a biochemical cue comprises a motif capable of entailing cell adhesion to a protein. In some embodiments, the protein comprises one amino acid sequence capable of functioning as a biochemical cue. In other embodiments, the protein comprises greater than one e.g., at least two, at least three, at least four, at least five, at least six, at least seven, at least eight, at least nine or at least ten or more amino acid sequences capable of functioning as a biochemical cue, for example, the protein can comprise 2, 3, 4, 5, 6, 7, 8, 9 or 10 amino acid sequences capable of functioning as a biochemical cue. In an embodiment, the protein comprises three amino acid sequences capable of functioning as a biochemical cue. In embodiments wherein the protein comprises at least two amino acid sequences capable of functioning as biochemical cues, the sequences can be the same or different. In an embodiment, the protein comprises at least two different amino acid sequences capable of functioning as biochemical cues. For example, in an embodiment, the protein comprises at least two different amino acid sequences capable of functioning as a biochemical cue, the first amino acid sequence having at least 90%, at least 95% or at least 99% sequence identity to the amino acid sequence set forth in SEQ ID NO:2 and the second amino acid sequence having at least 90%, at least 95% or at least 99% sequence identity to the amino acid sequence set forth in SEQ ID NO: 3. In another embodiment, the protein comprises at least two different amino acid sequences capable of functioning as a biochemical cue, the first amino acid sequence having at least 90% sequence identity to the amino acid sequence set forth in SEQ ID NO:2 and the second amino acid sequence having at least 90% sequence identity to the amino acid sequence set forth in SEQ ID NO:3. In a further embodiment, the first amino acid sequence has the amino acid sequence set forth in SEQ ID NO:2 and the second amino acid sequence has the amino acid sequence set forth in SEQ ID NO:3. In an embodiment, the protein comprises two copies of the amino acid sequence set forth in SEQ ID NO:3 or having at least 90%, at least 95% or at least 99% sequence identity to the amino acid sequence set forth in SEQ ID NO:3 and one copy of the amino acid sequence set forth in SEQ ID NO:2 or having at least 90%, at least 95% or at least 99% sequence identity to the amino acid sequence set forth in SEQ ID NO:2.

In an embodiment, the protein further comprises at least one folded globular domain. In an embodiment, the at least one folded globular domain comprises ferredoxin-like folds.

In an embodiment, the at least one folded globular domain comprises repeating motifs having at least 90% sequence identity to the amino acid sequence set forth in SEQ ID NO:1. In an embodiment, the at least one folded globular domain has been engineered to comprise a cysteine moiety. For example, in an embodiment, the at least one folded globular domain comprises (Cys-FL)ₓ where x is the number of protein repeat units and x is at least 4. In an embodiment, x is an integer of from 4 to 10. In another embodiment, x is an integer of from 4 to 8. In another embodiment, x is an integer of from 4 to 6. In a further embodiment, x is 4. In an embodiment, the protein comprises an unstructured protein sequence positioned between two folded globular domains. In another embodiment, the unstructured protein sequence comprises one of the at least one amino acid sequences capable of functioning as a biochemical cue.

In an embodiment, the protein comprises an amino acid sequence having at least 90%, at least 95% or at least 99% sequence identity to the amino acid sequence set forth in SEQ ID NO:10. In another embodiment, the protein comprises an amino acid sequence having at least 90% sequence identity to the amino acid sequence set forth in SEQ ID NO:10. In a further embodiment, the protein comprises the amino acid sequence set forth in SEQ ID NO: 10. In an embodiment, the protein is Fn-(Cys-FL)₄-RGD-(Cys-FL)₄-RGD.

The present disclosure also includes a protein hydrogel prepared by a method of preparing a protein hydrogel as described herein.

In some embodiments, the protein hydrogel of the present disclosure or prepared by a method for preparing a protein hydrogel of the present disclosure is capable of acting as an extracellular matrix for stem and/or progenitor cell differentiation.

The present disclosure also includes a use of a protein hydrogel of the present disclosure or prepared by a method of preparing a protein hydrogel of the present disclosure in tissue repair. The present disclosure also includes a protein hydrogel of the present disclosure or prepared by a method of preparing a protein hydrogel of the present disclosure for use in tissue repair. The present disclosure also includes a method of repairing tissue comprising applying a protein hydrogel of the present disclosure or prepared by a method of preparing a protein hydrogel of the present disclosure to the tissue in need thereof.

The present disclosure also includes a use of a protein hydrogel of the present disclosure or prepared by a method of preparing a protein hydrogel of the present disclosure in cartilage repair, bone repair or combinations thereof. The present disclosure also includes a protein hydrogel of the present disclosure or prepared by a method of preparing a protein hydrogel of the present disclosure for use in cartilage repair, bone repair or combinations thereof. The present disclosure also includes a method of repairing cartilage, bone or combinations thereof comprising applying a protein hydrogel of the present disclosure or prepared by a method of preparing a protein hydrogel of the present disclosure to the cartilage, bone or combinations thereof in need thereof. In an embodiment, the cartilage, bone or combinations thereof comprises a defect and the applying comprises implanting the protein hydrogel in the defect. In an embodiment, the cartilage, bone or combinations thereof is in a load-bearing joint such as a knee, wrist, ankle, elbow, shoulder, spine or hip.

The present disclosure also includes a use of a protein hydrogel of the present disclosure or prepared by a method of preparing a protein hydrogel of the present disclosure in repairing an osteochondral defect. The present disclosure also includes a protein hydrogel of the present disclosure or prepared by a method of preparing a protein hydrogel of the present disclosure for use in repairing an osteochondral defect. The present disclosure also includes a method of repairing an osteochondral defect comprising applying a protein hydrogel of the present disclosure or prepared by a method of preparing a protein hydrogel of the present disclosure to the osteochondral defect. In an embodiment, the applying comprises implanting the protein hydrogel in the osteochondral defect. In an embodiment, the method or use further comprises microfracture of subchondral bone prior to implantation of the protein hydrogel in the osteochondral defect. In an embodiment, the osteochondral defect is in a load-bearing joint such as a knee, wrist, ankle, elbow, shoulder, spine or hip.

The terms “repairing” and the like as used herein means at least partial repair such that beneficial or desired results, including clinical results are obtained in a subject. The term “repairing” and the like as used herein may include replacement and/or regeneration.

A person skilled in the art having regard to the present disclosure would readily appreciate that the selection of a suitable protein hydrogel of the present disclosure or prepared by a method for preparing a protein hydrogel of the present disclosure may depend, for example, on the nature of the method or use, and could readily select a suitable protein hydrogel accordingly. For example, it was surprisingly found that the protein hydrogel with a stiffness closer to that of cartilage led to worse repair efficacy due to its much slower degradation kinetics. Accordingly, in an embodiment of the methods and uses in repairing an osteochondral defect, the protein hydrogel is suitably degraded e.g., at least substantially degraded within a suitable time, e.g., eight weeks after application/implantation. In an embodiment of the methods and uses in repairing cartilage, bone or combinations thereof and/or repairing an osteochondral defect, the protein hydrogel has a compressive modulus of at least 0.1 MPa, for example, a compressive modulus of about 0.2 MPa to about 0.5 MPa, about 0.2 MPa to about 0.3 MPa or about 0.2 MPa, wherein the compressive modulus is measured as described herein.

It will also be appreciated by a person skilled in the art that embodiments relating to such methods and uses may be varied as described herein, as suitable, for embodiments relating to the protein hydrogels and methods for their preparation of the present disclosure.

The present disclosure also includes a synthetic protein comprising an amino acid sequence having at least 90%, at 95% or at least 99% sequence identity to the amino acid sequence set forth in SEQ ID NO:10. The present disclosure also includes a synthetic protein comprising an amino acid sequence having at least 90% sequence identity to the amino acid sequence set forth in SEQ ID NO:10. The present disclosure also includes a synthetic protein having the amino acid sequence set forth in SEQ ID NO:10. In an embodiment, the synthetic protein is Fn-(Cys-FL)₄-RGD-(Cys-FL)₄-RGD. The present disclosure also includes a use of such a synthetic protein in preparing a protein hydrogel. In an embodiment, the use is in a method of preparing a protein hydrogel of the present disclosure.

The following are non-limiting examples of the present disclosure:

### EXAMPLES

### Example 1

Physical entanglement was used to significantly stiffen protein-based hydrogels without compromising their toughness. By introducing physical entanglements into the network of soft hydrogels made of folded elastomeric proteins, a highly tough and stiff protein hydrogel was engineered, effectively converting a soft biomaterial to a stiff and tough material exhibiting mechanical properties close to those of cartilage. The resultant protein hydrogels exhibited outstanding mechanical properties that seamlessly combined mutually incompatible properties, including high stiffness, high toughness, fast recovery and ultrahigh compressive strength. The hydrogel showed a Young's modulus of about 0.7 MPa and tensile toughness of 250 KJ/m³; and about 1.7 MPa in compressive modulus and compressive toughness of 3.2 MJ/m³, and can withstand a compression stress of greater than 60 MPa without failure, amongst the highest compressive strength achieved by a hydrogel. This opens a new and general avenue towards engineering stiff and tough protein-based biomaterials, which will find applications in biomedical engineering, such as osteochondral defect repair, as well as applications in material sciences and engineering.

### I. Materials and Methods

Methods Summary: The (FL)₈, (FL-M23C)₈ and Fn-(Cys-FL)₄-RGD-(Cys-FL)₄-RGD polyproteins were engineered using previously published protocols (Fang & Li, 2012). Protein hydrogels were constructed using a previously published photochemical crosslinking strategy. For the DC hydrogel, the photochemical crosslinking was carried out in 7 M GdHCl solution. For the NC hydrogel, the photochemical crosslinking was carried out in phosphate-buffered saline (PBS) solution. However, this method can be readily adapted to other crosslinking chemistry. For example, hydrogels were also successfully prepared using disulfide crosslinking. Tensile and compression tests were performed on an Instron-5500R universal testing system. The local slope at 15% strain on the loading curve was taken as the modulus for both tensile and compression tests. The toughness was calculated as the area between the loading curves at a given strain using custom-written software in IgorPro. The animal study was carried out in compliance with the regulations and guidelines of the Ethics Committee of Drum Tower Hospital affiliated to the Medical School of Nanjing University and conducted according to the Institutional Animal Care and Use Committee (IACUC) guidelines.

Protein engineering: FL domain is a redesigned variant of Di-I_5 (PDB code: 2KL8) (Koga et al., 2012; Fang et al., 2013). The amino acid sequence of FL is: MGEFDIRFRT DDDEQFEKVL KEMNRRARKD AGTVTYTRDG NDFEIRITGI SEQNRKELAK EVERLAKEQN ITVTYTERGS LE (SEQ ID NO: 1). The genes of the polyprotein ferredoxin-like proteins (FL)₈, (FL-M23C)₈, (FL)₁₆, Fn-(Cys-FL)₄-RGD-(Cys-FL)₄-RGD and (FL-M23C)₈-Fn were constructed following standard and well-established molecular biology methods as reported previously (Lv et al., 2010). Fn is the third FnIII domain from human extracellular matrix protein tenascin and has the sequence SRLDAPSQIEVKDVTDTTALITWFKPLAEIDGIELTYGIKDVPGDRTTIDLTEDENQY SIGNLKPDTEYEVSLISRRGDMSSNPAKETFTT (SEQ ID NO:2), and RGD is a 17 aa residue long sequence, TVYAVTGRGDSPASSRS (SEQ ID NO:3) derived from cell adhesion protein fibronectin that contains the integrin-binding RGD (Arginine-Glycine-Glutamate) motif. Other polyproteins, (GB1)₈, (NuG2)₈, GRG₅RG₄R, N₄RN₄RNR and (GA)₈, were constructed following the same method. Amino acid sequences of the elastomeric proteins engineered are shown in Table 1.

TABLE 1
Amino acid sequence of elastomeric proteins engineered.

| Protein | Amino Acid Sequence |
|---|---|
| (FL)₈ | MRGSHHHHHHGS(MGEFDIRFRTDDDEQFEKVLKEMNRRARKDAGTVT |
| | YTRDGNDFEIRITGISEQNRKELAKEVERLAKEQNITVTYTERGSLERS)₈ (SEQ ID NO: 4) |
| (FL-M23C)₈ | MRGSHHHHHHGS(MGEFDIRFRTDDDEQFEKVLKECNRRARKDAGTVTY |
| | TRDGNDFEIRITGISEQNRKELAKEVERLAKEQNITVTYTERGSLERS)₈ (SEQ ID NO: 5) |
| (NuG2)₈ | MRGSHHHHHHGS(MDTYKLVIVLNGTTFTYTTEAVDAATAEKVFKQYA |
| | NDNGVDGEWTYDDATKTFTVTERS)₈ (SEQ ID NO: 6) |
| (GB1)₈ | MRGSHHHHHHGS(MDTYKLILNGKTLKGETTTEAVDAATAEKVFKQYA |
| | NDNGVDGEWTYDDATKTFTVTERS)₈ (SEQ ID NO: 7) |
| GRG₅RG₄R | MRGSHHHHHHGSMDTYKLILNGKTLKGETTTEAVDAATAEKVFKQYAN |
| | DNGVDGEWTYDDATKTFTVTERSGGRPSDSYGAPGGGNRS(MDTYKLIL |
| | NGKTLKGETTTEAVDAATAEKVFKQYANDNGVDGEWTYDDATKTFTV |
| | TERS)₅GGRPSDSYGAPGGGNRS(MDTYKLILNGKTLKGETTTEAVDAATA |
| | EKVFKQYANDNGVDGEWTYDDATKTFTVTERS)₄GGRPSDSYGAPGGGN (SEQ ID NO: 8) |
| NRN₄RN₄R | MRGSHHHHHHGSMDTYKLVIVLNGTTFTYTTEAVDAATAEKVFKQYAN |
| | DNGVDGEWTYDDATKTFTVTERSGGRPSDSYGAPGGGNRS(MDTYKLVI |
| | VLNGTTFTYTTEAVDAATAEKVFKQYANDNGVDGEWTYDDATKTFTVT |
| | ERS)₄GGRPSDSYGAPGGGNRS(MDTYKLVIVLNGTTFTYTTEAVDAATAE |
| | KVFKQYANDNGVDGEWTYDDATKTFTVTERS)₄GGRPSDSYGAPGGGN (SEQ ID NO: 9) |
| Fn-(Cys- | MRGSHHHHHHGSRLDAPSQIEVKDVTDTTALITWFKPLAEIDGIELTYGIK |
| FL)₄-RGD- | DVPGDRTTIDLTEDENQYSIGNLKPDTEYEVSLISRRGDMSSNPAKETFTT |
| (Cys-FL)₄- | RS(CMGEFDIRFRTDDDEQFEKVLKEMNRRARKDAGTVTYTRDGNDFEIR |
| RGD | ITGISEQNRKELAKEVERLAKEQNITVTYTERGSLRS)₄TVYAVTGRGDSPA |
| | SSRS(CMGEFDIRFRTDDDEQFEKVLKEMNRRARKDAGTVTYTRDGNDF |
| | EIRITGISEQNRKELAKEVERLAKEQNITVTYTERGSLRS)₄TVYAVTGRGD |
| | SPASS (SEQ ID NO: 10) |
| (FL-M23C)₈- | MRGSHHHHHHGS(MGEFDIRFRTDDDEQFEKVLKECNRRARKDAGTVTY |
| Fn | TRDGNDFEIRITGISEQNRKELAKEVERLAKEQNITVTYTERGSLERS)8SRL |
| | DAPSQIEVKDVTDTTALITWFKPLAEIDGIELTYGIKDVPGDRTTIDLTEDE |
| | NQYSIGNLKPDTEYEVSLISRRGDMSSNPAKETFTT (SEQ ID NO: 11) |

Polyprotein genes were inserted into the vector pQE80L for protein expression in E. coli strain DH5α. Seeding culture was allowed to grow overnight in 10 mL 2.5% Luria-Bertani broth (LB) medium containing 100 mg/L ampicillin. The overnight culture was used to inoculate 1 L of LB medium which was grown at 37° C. and 225 rpm for 3 hours to reach an OD600 of about 0.8. Protein expression was induced with 1 mM isopropyl-1-β-D-thiogalactoside (IPTG) and continued at 37° C. for 4 hours. The cells were harvested by centrifugation at 4000 rpm for 10 mins at 4° C. and then frozen at −80° C. For polyprotein purification, cells were thawed and resuspended in 1× phosphate-buffered saline (PBS) and lysed by incubation with 1 mg/mL lysozyme for 30 mins. Nucleic acids were removed by adding 0.1 mg/mL of both DNase and RNase. The supernatant with soluble protein was collected after centrifuging the cell mixture at 12000 rpm for 60 mins. The soluble His₆-tagged protein was purified using a Co²⁺ affinity column. The yields of (FL)₄, (FL)₈, (FL-M23C)₈, and (FL)₁₆ were approximately 90 mg, 80 mg, 80 mg and 45 mg respectively per liter of bacterial culture. Purified proteins were dialyzed extensively against deionized water for 2 days to remove residual NaCl, imidazole, and phosphate. Then the protein solution was filtered and lyophilized, and stored at room temperature until use. Bovine serum albumin (BSA) lyophilized powder was purchased from Sigma-Aldrich.

Single-molecule optical tweezers measurements: Single-molecule optical tweezers measurements were carried out using a MiniTweezers setup as previously described (http://tweezerslab.unipr.it; Lei et al., 2017). Sample preparation including the protein-DNA construct formation and force measurement protocols was adapted from protocols described previously (Lei et al., 2017). Force-distance curves of the protein-DNA construct were obtained using constant velocity pulling protocol.

Hydrogel preparation and dimension measurements: Lyophilized (FL)₈ protein was dissolved in PBS and 7M GdHCl (with a concentration of 20%) to obtain the folded and unfolded (FL)₈ protein solutions. The viscosity of protein solution was determined by using a TA Instruments Discovery HR-2 equipped with a 20 mm flat plate. (FL)₈ protein hydrogels were prepared using two different gelation methods. The NC (native crosslinking) hydrogels were prepared in native state by dissolving and gelating proteins in 1×PBS. After gelation, N-NC hydrogels were soaked in PBS, while the D-NC hydrogels were stored in 1×PBS containing 7M guanidine-hydrochloride (GdHCl) and achieved swelling equilibrium. The DC (denatured crosslinking) hydrogels (D-DC and N-DC) were prepared by dissolving the lyophilized (FL)₈ in 7M GdHCl for 2 hrs before use. The denatured protein solution was crosslinked into hydrogels and equilibrated in 7M GdHCl to obtain D-DC hydrogels, while N-DC hydrogels were renatured in PBS on a rocker by changing fresh PBS ten times over the course of 1 day until reaching equilibrium.

Gelation of (FL)₈, (FL-M23C)₈ and (FL)₁₆ were based on a photochemical crosslinking strategy described previously (Fang et al., 2013; Lv et al., 2010; Fancy & Kodadek, 1999; Elvin et al., 2005). To prepare 20% (w/v) hydrogels, lyophilized proteins (200 mg/mL) were re-dissolved in PBS (D-NC and N-NC) or 7M GdHCl in PBS (D-DC and N-DC) respectively. A typical crosslinking reaction mixture contained 200 mg/mL of polyprotein, 50 mM ammonium persulfate (APS) and 200 μM [Ru(bpy)₃]Cl₂. The protein mixture was cast into a custom-made plexiglass ring-shaped mold (dᵢₙ=8 mm, dₒᵤₜ=10 mm, h=3 mm), and was exposed to a 200 W fiber optical white light source placed 10 cm above the mold for 10 min at room temperature. After gelation was complete, the hydrogel sample was carefully taken out of the mold. After the ring-shaped hydrogels were stored in the desired buffers for 3 hrs (N-DC gels for 24 hrs), the outer diameter, thickness, width and weight of all ring-shape equilibrated swollen/deswelling samples were measured before tensile tests. For compressive tests and SEM imaging, the hydrogels were prepared in a cylindrical shape following the same gelation procedures. The hydrogels preparation and the tensile (E) and compressive (Y) moduli measurements of (GB1)₈, (NuG2)₈, GRG₅RG₄R, NRN₄RN₄R, (GA)₈ and BSA followed the same procedures.

A disulfide-based oxidation method was also used to prepare DC hydrogels of (FL-M23C)₈ and the DC hydrogels of Fn-(Cys-FL)₄-RGD-(Cys-FL)₄-RGD (FLRGD), and (FL-M23C)₈-Fn. Briefly, 20% protein solution in 7M GdHCl was cast into a custom-made plexiglass mold and let it stand overnight to allow air oxidation. After the gelation, the D-DC hydrogels were carefully taken out of the mold and renatured in PBS on a rocker by changing fresh PBS ten times over the course of 1 day until reaching equilibrium.

A soft FLRGD/gelatin hydrogel was prepared using NHS-EDC crosslinking chemistry. In particular, an aqueous solution of FLRGD/gelatin (with a concentration of 5% FLRGD and 5% gelatin) was prepared at 40° C., and then let stand at room temperature for 2 hrs to allow gelatin to form a physically crosslinked hydrogel. The hydrogel was then chemically crosslinked in PBS using 15 mM N-(3-dimethylaminopropyl)-N-ethylcarbodiimide (EDC)/6 mM N-hydroxysuccinimide (NHS) for 1 hr.

Tensile tests: Tensile tests were performed using an Instron-5500R tensometer with a custom-made force gauge and 5-N load transducer. The ring-shaped hydrogel specimen was stretched and relaxed in PBS (N-DC and N-NC) or 7 M GdHCl in PBS (D-DC and D-NC) at constant temperature (25° C.) without special preconditioning. The stress was calculated by dividing the load by the initial cross-sectional area of the hydrogel sample. The Young's modulus, breaking strain, and energy dissipation were measured using an extension rate of 25 mm/min. Young's modulus of the sample was measured at a strain between 10-15%. Toughness was determined by integrating stress-strain curves where specimens were loaded directly to failure. Energy dissipation was calculated by integrating loop area between stretching and relaxing stress-strain curves. In hysteresis recovery experiments, a pulling rate of 200 mm/min was used. The same ring sample was stretched and relaxed with various time intervals. For technical considerations, ring-shaped specimens were used for tensile testing. Tensile testing of rings of material was conducted to minimize difficulties that arise from gripping soft materials. Because the test strains are large in these experiments, gripped material would thin substantially upon stretching, so the material would need to be clamped so tightly that it would fail at the grips. Self-adjusting pneumatic grips that automatically adjust for material thinning are designed for materials much stiffer than the present polyproteins and would have the same problem of material failure or slippage. Previously published methods were followed for testing arterial elastin rings (Lillie et al., 1994) and protein-based biomaterials (Lv et al., 2010) to avoid these problems.

Compression tests: Uniaxial compression tests were performed on cylinder-shaped hydrogels that were swollen to equilibrium using the Instron-5500R with 5000-N load transducer in air at room temperature. The dimensions (height (h₀) and diameter (d₀)) of the equilibrated N-DC and N-NC (FL)₈ hydrogel samples were: h₀: 3.0 mm and d₀: 6.5 mm for the N-DC hydrogel, and h₀: 5.0 mm and d₀: 5.0 mm for the N-NC hydrogel. The gel was put on the lower plate, while the upper plate approached the sample slowly until a rise in force was detected, indicating contact between the plate and the gel. The stress was calculated by dividing the load by the initial cross-sectional area of the hydrogel sample. Unless a different rate is stated, the gel was compressed and relaxed at a compression speed of 2 mm/min. No water was squeezed out of the gels during compression. The compressive modulus was measured at a strain of 10-20%. The maximum stress and strain were determined at failure points, where the first crack in the gel was observed. Energy dissipation was calculated by integrating loop area between compressing and relaxing stress-strain curves (n=7). In hysteresis recovery experiments, a compression rate of 100 mm/min was used.

Characterization of dityrosine cross-links in hydrogels by acid hydrolysis-fluorescence method: The degree of dityrosine crosslinking in both NC and DC (FL)₈ hydrogels was characterized following a well-established fluorimetry method (Fang & Li, 2012). Dityrosine emits at a wavelength of 410 nm when excited at 315 nm. For quantification of the dityrosine and dityrosine-like compounds generated in NC and DC (FL)₈ hydrogels, a well-established fluorescence standard curve method (Fang & Li, 2012) was followed. Typically, 20% (w/v) hydrogel samples (about 25 mg) were reacted with 100 μL HCl (6 N) in a sealed 1.5 mL centrifuge tube in a metal heat block at 105° C. for 2 hrs to achieve full hydrolysis of the peptide bonds. Then, 100 μL of acid hydrolysis product was transferred into a new 1.5 mL centrifuge tube and neutralized by 10 μL NaOH (5 M). Next, 100 mM Na₂CO₃—NaHCO₃buffer (pH 9.9) was added to a final volume of 1 mL. Fluorescence spectra of the samples were measured by a Varian Cary Eclipse fluorescence spectrophotometer. According to the fluorescence-concentration standard calibration curve of dityrosine, the yield of dityrosine and dityrosine-like products in the hydrogel was then determined (n=8).

Cysteine shotgun fluorescence labeling by IAEDANS and fluorescence measurements: DC and NC (FL-M23C)₈ hydrogels for cysteine shotgun labeling were prepared with the same protein concentration and gel preparation procedures as the wild-type (FL)₈. The labeling reaction was performed in the dark at room temperature for 3 hrs in PBS buffer (pH 7.4) containing 5 mM tris(2-carboxyethyl)phosphine (TCEP) and 2 mM 5-((2-[(iodoacetyl)amino]ethyl)amino)naphthalene-1-sulfonic acid (IAEDANS). As a control, D-DC and D-NC (FL-M23C)₈ hydrogels incubated in 7 M GdHCl (containing 5 mM TCEP, pH 7.4) were also labeled using IAEDANS. Then all hydrogels were transferred to PBS buffer containing 5 mM β-mercaptoethanol to quench the reaction. To remove excess labeling dye, additional PBS buffer (containing 5 mM β-mercaptoethanol) was added, and changed five times over the course of 5 hrs until fluorescence could no longer be detected in the buffer solution. To quantify the fluorescence intensity of IAEDANS labeled hydrogels, the hydrogels were digested with trypsin at 37° C. for 5 hrs. The digestion reaction contained 5% trypsin (relative to the hydrogel weight), 25 mM NH₄HCO₃, 10 mM CaCl₂), 1 M GdHCl and 10 mM dithiothreitol. Unlabeled (FL-M23C)₈ hydrogel was digested in the same way to serve as a negative control. After digestion, 50 μL of the digested mixture was diluted to 3 mL using PBS buffer. The fluorescence emission spectrum was measured by a Varian Cary Eclipse fluorescence spectrometer using an excitation wavelength of 336 nm and emission at 490 nm was monitored. An IAEDANS standard calibration curve was also created, covering linear concentration range of 0-60 μM (n=8).

Swelling ratio and water content measurements: For swelling ratio and water content measurements, ring-shaped hydrogels were weighed immediately after being taken out of the mold, and the weight was recorded as mᵢ. The swollen weight mₛ was recorded after gently removing excess buffer from equilibrated hydrogels. To measure dry weight of the gels (m_{dry}), the hydrogels were firstly immersed in deionized water to remove extra salts, then lyophilized for 2 days and dried in a 70° C. incubator for 1 day. The swelling ratio (r) of the hydrogels was calculated using the following formula:

$$r = \frac{{m}_{s} - {m}_{i}}{{m}_{i}} \times 100 \% .$$

The water content (w) was determined by the dried gel (m_{dry}) and the equilibrated gel (ms) (measured specimens, n=4):

$$w = ( 1 - \frac{{m}_{dry}}{{m}_{s}} ) \times 100 \% .$$

Scanning electron microscopy (SEM) imaging: 20% (w/v) D-NC and N-NC (FL)₈ hydrogel samples were prepared for SEM imaging using a Hitachi S4700 scanning electron microscope. The samples were then shock-frozen in liquid nitrogen, and quickly transferred to a freeze drier where they were lyophilized for 24 hrs. Lyophilized samples were then carefully fractured in liquid nitrogen, and fixed on aluminum stubs. The sample surface was coated by 8 nm of gold prior to SEM measurements.

Cytotoxicity assay: N-DC-FLRGD hydrogels were prepared into a disk shape (with 8 mm in diameter and 1 mm in height) by air oxygen oxidation overnight, and allowed to reach swell equilibrium in PBS. After being sterilized by UV irradiation, the hydrogels were put into 24-well cell culture plates and MC3T3-E1 cells were seeded on the hydrogels and wells at a density of 1×10⁵ per well. Growth medium (GM), α-MEM medium (Gibco, USA) supplemented with 10% fetal bovine serum (FBS; Gibco, USA) and 1% penicillin/streptomycin solution (P/S; Gibco, USA) were used in all the incubations. After culturing for 48 hours, the cells were assayed with live/dead assay kits. Briefly, the cells and samples were washed with PBS and then fixed with paraformaldehyde for 5 minutes. After washing with PBS again, cells were dyed with Calcein AM and propidium iodide (PI) for 45 minutes and then observed under a laser scanning confocal microscope (Olympus 141 FV3000, Tokyo, Japan).

Cell differentiation: MC3T3-E1 cells were cultured on hydrogels and cell culture dishes as described above. For the gel and control groups, osteogenic differentiation medium (OM) (GM supplemented with 0.1 μM dexamethasone (Gibco, USA), 50 μg mL-1 ascorbic acid (Gibco, USA), and 10 mM β-glycerophosphate (Gibco, USA)) were used in the incubation. For the blank group, growth medium (GM), α-MEM medium (Gibco, USA) supplemented with 10% fetal bovine serum (FBS; Gibco, USA) and 1% penicillin/streptomycin solution (P/S; Gibco, USA) were used in the incubation. After culturing for 5 or 10 days, the cells were fixed in 2% (vol/vol) paraformaldehyde for 30 min and then treated with 0.1% Triton™ X-100 for 15 min. After blocking in 5% (wt/vol) BSA for 1 hour to minimize nonspecific binding, anti-Col I (cat: 14695, Proteintech, USA) or anti-RUNX2 (cat: ab23981, Abcam, China) primary antibodies diluted with antibody dilution buffer were added to the fixed cells and incubated overnight at 4° C. Then, the primary antibody solution was decanted, and the dish was immediately washed with PBS 3×5 min. After being rinsed with PBST (0.5 wt % Tween™-20 in PBS) 3 times, the secondary antibody (Alexa Fluor 488 donkey anti-rabbit lgG (H+L)) in PBS was added to the dish, and the cells were incubated for 60 min at room temperature. Then, the secondary antibody was decanted, and the cells were washed with PBST 3×5 min. All images were obtained using a Nikon-Ti-2U fluorescence microscope (Nikon, Japan).

qRT-PCR analysis: Total RNA was isolated from the cells cultured for 5 and 10 days with the RNA-Quick Purification Kit (Yishan Biotech, Shanghai, China) and cDNA was generated with a HiScriptIIQ RT SuperMix for qPCR (Vazyme Biotech, Nanjing, China). Then the results were analyzed using a ChamQ™ SYBR Color qPCR Master Mix (Vazyme Biotech). The amplification and detection were carried out on a LightCycler 480-II (Roche, Mannheim, Germany). The primer sequences are listed in Table 2.

TABLE 2
Sense and antisense sequences of the primers used
for quantitative RT-PCR reactions.

| Gene | Forward | Reverse |
|---|---|---|
| GAPDH | AGGTCGGTGTGAACGGATTTG | TGTAGACCATGTAGTTGAGGTCA |
| ALP | CCAACTCTTTTGTGCCAGAGA | GGCTACATTGGTGTTGAGCTTTT |
| collagen I | GCTCCTCTTAGGGGCCACT | CCACGTCTCACCATTGGGG |
| RUNX2 | GACTGTGGTTACCGTCATGGC | ACTTGGTTTTTCATAACAGCGGA |
| OPN | GCAATAAGGTAGTGAACAGACTCC | CCATAGATGCGTTTGTAGGCGG |

The levels of the target transcript were normalized to that of the internal reference (2-ΔΔCT method). Statistical significance was determined using Student's t-test, or one-way ANOVA accordingly. Statistical significance was set to a P value <0.05.

In vivo study: The animal study was carried out in compliance with the regulations and guidelines of the Ethics Committee of Drum Tower Hospital affiliated to the Medical School of Nanjing University and conducted according to the Institutional Animal Care and Use Committee (IACUC) guidelines. A total of 36 female New Zealand white rabbits with a bodyweight of 2.5 kg were used in the study. For 18 animals, one knee (chosen randomly) was used as a blank control, and the other was treated with highly stiff (FL-M23C)₈-Fn hydrogel implants. For the other 18 animals, one knee was randomly selected to implant with soft FLRGD/gelatin hydrogels, and the other was implanted with the stiff FLRGD hydrogels. A full thickness osteochondral defect with a height of 4 mm and a diameter of 5 mm was made in the center of the trochlear groove. The defects in the hydrogel treated groups were implanted with hydrogel cylinders, and the defects in the control group remained blank. Cefuroxime sodium was injected intramuscularly for 3 days after operation to avoid infection. For each time point (week 4, 8 and 12 post-operation), 12 animals were randomly selected and sacrificed to evaluate the chondrogenic and osteogenesis capacity. The major organs were obtained at each time point to evaluate the biocompatibility of the hydrogels.

Statistical design of the animal experiments: To determine the sample size of the animal studies needed for the osteochondral defect repair, a statistical analysis was carried out. Based on the standard deviation of the experimental data from previous experiments as well as those from the literature, it was found that a sample size of 6 will guarantee a statistical power greater than 80%. To minimize the number of animals used in the experiments, a sample size of 6 was chosen for each experiment.

Macroscopic evaluation: The International Cartilage Repair Society (ICRS) macroscopic scoring was used to assess the macroscopic appearance of the repair tissue (van den Borne et al., 2007). The scoring criteria is shown in Table 3, including the defect filling, integration to native cartilage, and repair tissue surface topography.

TABLE 3
ICRS scoring system.

| Cartilage repair evaluation | Points |
|---|---|
| Degree of defect repair | |
| In level with surrounding cartilage | 4 |
| 75% repair of defect depth | 3 |
| 50% repair of defect depth | 2 |
| 25% repair of defect depth | 1 |
| 0% repair of defect depth | 0 |
| Integration to border zone | |
| Complete integration with surrounding cartilage | 4 |
| Demarcating border <1 mm | 3 |
| ¾th of graft integrated, ¼th with a notable border >1 mm width | 2 |
| ½ of graft integrated with surrounding cartilage, ½ with a notable border >1 mm | 1 |
| From no contact to ¼th of graft integrated with surrounding cartilage | 0 |
| Macroscopic appearance | |
| Intact smooth surface | 4 |
| Fibrillated surface | 3 |
| Small, scattered fissures or cracks | 2 |
| Several, small or few but large fissures | 1 |
| Total degeneration of grafted area | 0 |
| Overall repair assessment | |
| Grade I: normal | 12 |
| Grade II: nearly normal | 11-8 |
| Grade III: abnormal | 7-4 |
| Grade IV: severely abnormal | 3-1 |

Imaging examination and histological evaluation: The harvested samples were fixed in 10% formalin for 24 hours before the imaging examination. The XM micro CT system (Hiscan, China) and the 9.4T Bruker Biospec 94/20 USR micro MRI system (Bruker, Germany) were employed to perform the CT and MRI tests. Then, the samples were decalcified in 15% EDTA for 28 days, embedded in paraffin, and cut into 5 μm thick sections. These were then stained with haematoxylin & Eosin (H&E), toluidine blue, and Safranin O. The histological results were assessed using the O'Driscoll scoring system (O'Driscoll et al., 1988), the scoring criteria for which is shown in Table 4. All sections were observed using a microscope equipped with a CCD camera (Olympus, Japan).

TABLE 4
O'Driscoll scoring system.

| Histological evaluation | Points |
|---|---|
| Nature of the predominant tissue | |
| Cellular morphology | 4 |
| Hyaline articular cartilage | |
| Incompletely differentiated mesenchyme | 2 |
| Fibrous tissue or bone | 0 |
| Safranin O staining of the matrix | |
| Normal or nearly normal | 3 |
| Moderate | 2 |
| Slight | 1 |
| None | 0 |
| Structural characteristics | |
| Surface regularity | |
| Smooth and intact | 3 |
| Superficial horizontal lamination | 2 |
| Fissures - 25 to 100 per cent of thickness | 1 |
| Severe disruption, including fibrillation | 0 |
| Structural integrity | |
| Normal | 2 |
| Slight disruption, including cysts | 1 |
| Severe disintegration | 0 |
| Thickness | |
| 100% of normal adjacent cartilage | 2 |
| 50-100% of normal cartilage | 1 |
| 0-50% of normal cartilage | 0 |
| Bonding to adjacent cartilage | |
| Bonded at both ends of graft | 2 |
| Bonded at one end, or partially at both ends | 1 |
| Not bonded | 0 |
| Freedom from cellular changes of degeneration | |
| Hypocellularity | |
| Normal cellularity | 3 |
| Slight hypocellularity | 2 |
| Moderate hypocellularity | 1 |
| Severe hypocellularity | 0 |
| Chondrocyte clustering | |
| No clusters | 2 |
| <25% of the cells | 1 |
| 25-100% of the cells | 0 |
| Freedom from degenerative changes in adjacent cartilage | |
| Normal cellularity, no clusters, no staining | 3 |
| Normal cellularity, mild clusters, moderate staining | 2 |
| Mild or moderate hypocellularity, slight staining | 1 |
| Severe hypocellularity, poor or no staining | 0 |

Statistical analysis: The macroscopic and histological results were analyzed by three investigators who were blind to the groups. Unpaired Student's t-test was performed using SPSS 19.0 software (IBM Corp. Armonk, NY). P<0.05 was considered statistically significant. *: p<0.05; **: p<0.01 and ***: p<0.001.

Evaluation of immunological response of animals after hydrogel implantation: A total of 18 male Sprague Dawley (SD) rats with a body weight of 250 g were used in the test. The animals were randomly divided into 3 groups (6 rats for each group). Gelatin was used as the negative control group due to its good biosafety and low immunogenicity. For the gelatin and stiff FLRGD hydrogel groups, the gelatin or stiff FLRGD hydrogel discs with a diameter of 4 mm and a height of 1 mm were implanted subcutaneously. The blank group received the same surgical procedure but remained blank in the incision. Three rats from each group were randomly selected and sacrificed after implantation for 48 hours. The other rats were sacrificed after implantation for 7 days. The major organs and skins were obtained to evaluate the immunological response. In addition, the blood samples were also collected to test the liver function.

### II. Results & Discussion

In order to engineer highly stiff and tough protein hydrogels, a higher effective crosslinking density and an efficient microscopic mechanism for energy dissipation and fast recovery are essential and it was reasoned that if an efficient method can be developed to significantly improve the stiffness without increasing the brittleness of such soft protein biomaterials, one should be able to convert such soft protein hydrogels to materials with mechanical properties mimicking those of articular cartilage. Increasing the chemical crosslinking density of the protein hydrogel network cannot achieve the desired effect, as doing so will enhance stiffness but embrittle the hydrogel at the same time. A physical mechanism: chain entanglement was resorted to.

The feasibility to introduce chain entanglement into a soft protein hydrogel to significantly increase its stiffness without compromising its toughness was explored. For this purpose, the elastomeric polyprotein (FL)₈ was used, which is made up of eight tandem repeats of a de novo designed ferredoxin-like globular protein (FL) (Koga et al., 2012) (Table 1).

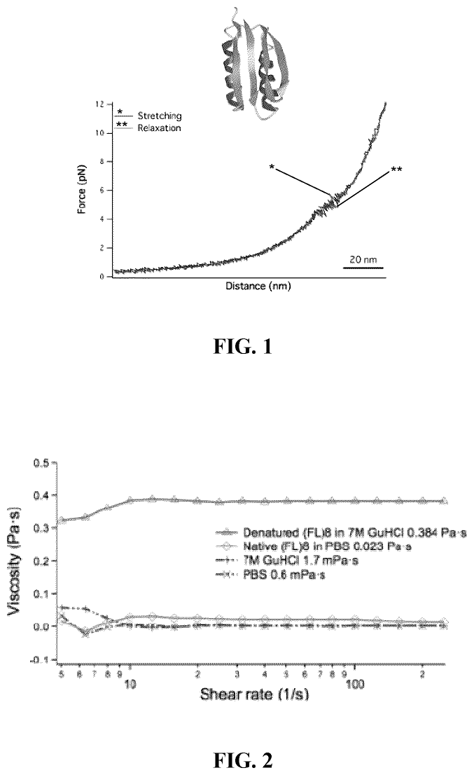

Single molecule optical tweezers experiments showed that FL is mechanically labile, and undergoes forced unfolding and refolding at about 5 pN (FIG. 1) (Fang et al., 2013). (FL)₈, which is a large tandem modular protein and can be expressed with a high yield in E. coli, was used to engineer highly stretchy and tough protein hydrogels, in which the forced-unfolding of FL domains served as a highly effective means in dissipating energy in the hydrogel (Fang et al., 2013). However, the (FL)₈ hydrogel is soft showing a Young's modulus of about 16 kPa (Fang et al., 2013). (FL)₈ was used as a model system to demonstrate the feasibility of using the same protein building blocks to achieve much enhanced mechanical stiffness while preserving its high toughness.

The molecular weight of (FL)₈ is about 80 kDa, but its contour length in its native state is only about 10 nm. Thus, there is no chain entanglement in the (FL)₈ hydrogel network. However, unfolded (FL)₈ is about 260 nm long, a typical length for polymers (260 nm corresponds to the length of polystyrene with a Mw of about 170 kDa). In the concentrated solution of unfolded (FL)₈, the unfolded polypeptide chains will overlap and likely entangle, as evidenced by its high viscosity (FIG. 2) (Colby, 2010).

It was reasoned that if unfolded (FL)₈ is crosslinked from its concentrated solution, chain entanglement could be trapped in the chemically crosslinked hydrogel network.

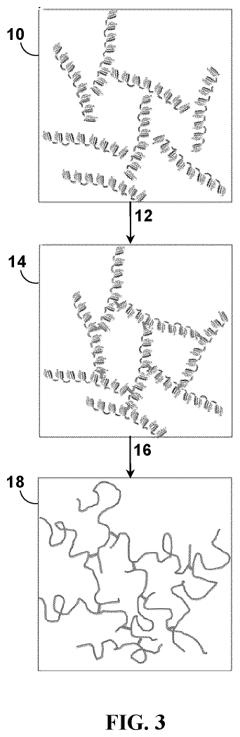

To test this, the well-developed [Ru(bpy)₃]²⁺-mediated photochemical crosslinking strategy (Lv et al., 2010; Elvin et al., 2005; Fancy & Kodadek, 1999; Partlow et al., 2016), which crosslinks two tyrosine residues in proximity into a dityrosine adduct, was employed to construct (FL)₈ hydrogels. The DC (denatured crosslinking) method was used to construct the denatured (FL)₈ hydrogels: a concentrated (200 mg/ml) solution of native (FL)₈ was denatured by using 7 M guanidine hydrochloride (GdHCl) to prepare a concentrated solution of unfolded (FL)₈, which was then photochemically crosslinked into a hydrogel. The as-prepared hydrogel was equilibrated in 7 M GdHCl to obtain the denatured DC hydrogel (referred to as the D-DC hydrogel). As a control, a denatured (FL)₈ hydrogel was also constructed that is free of chain entanglement using the NC (native crosslinking) method (FIG. 3). Referring to FIG. 3, the elastomeric protein (FL)₈ (top) was first dissolved in phosphate buffered saline (PBS) to a high concentration (about 200 mg/mL) to form native protein solution 10. Upon photochemical crosslinking 12, (FL); were crosslinked into a hydrogel network without chain entanglements, due to the short length of folded (FL)₈, resulting in the N-NC (native NC) hydrogel 14. When denatured 16 and equilibrated in 7M GdHCl, the (FL)₈ in the hydrogel network unfolded, and behaved as random coils. The resultant D-NC (denatured NC) hydrogel 18 is also free of chain entanglement.

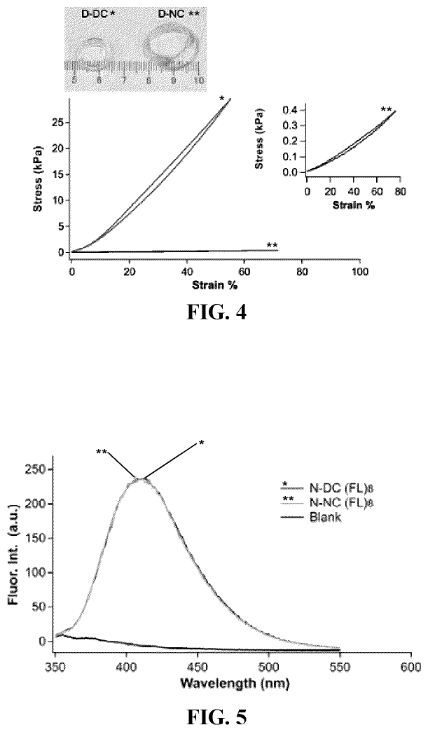

FIG. 4 shows the photographs of both D-DC (left) and D-NC (right) (FL)₈ hydrogels prepared using the same ring-shaped mold as well as their stress-strain curves. The D-DC hydrogel was self-standing and swelled to a much less degree than the D-NC hydrogel, while the D-NC ring-shaped hydrogel collapsed onto itself. The D-DC hydrogel displayed a Young's modulus of 56 kPa, significantly higher than that of D-NC hydrogel (about 1 kPa).

According to the affine network model of rubber elasticity theory and taking into account of the effect of dangling ends on the network elasticity (Treloar, 1979),

$$E = \frac{3 ρ RT}{{M}_{c}} ( 1 - \frac{2 {M}_{c}}{{M}_{n}} ) ,$$

where E is the Young's modulus of the hydrogel, p is the density of the polymer in the network, RT is the thermal energy, Mₙ is the number average molecular weight of the polymer, and M_{c} is the average molecular weight of the elastically effective strands. From this relationship, M_{c} can be determined and the total number of “effective” crosslinking points in the hydrogel network N_{eff} can then be calculated as

$${N}_{eff} = \frac{{M}_{n}}{{M}_{c}} \times n ,$$

where n is the number of moles of the polymer in the hydrogel. It was found that N_{eff} of the D-DC gel is about 1.6 times that of the D-NC gel. To examine if the difference in N_{eff} is due to the different number of chemical crosslinks N_{chem} (which is the number of dityrosine adducts) caused by the two different hydrogelation methods, N_{chem} was quantified by measuring the characteristic dityrosine fluorescence (FIG. 5) (Elvin et al., 2005; Fang & Li, 2012). The results showed that both hydrogels contained roughly the same N_{chem} (about 17% of the total number of tyrosine residues in FL domains were crosslinked into dityrosine adducts). Since the D-DC and D-NC hydrogels had the same N_{chem}, the same unfolded protein chain and the same protein chain-solvent interactions, the higher N_{eff} of the D-DC hydrogel must originate from the chain entanglements of unfolded (FL)₈ polypeptide chains in the D-DC hydrogel network.

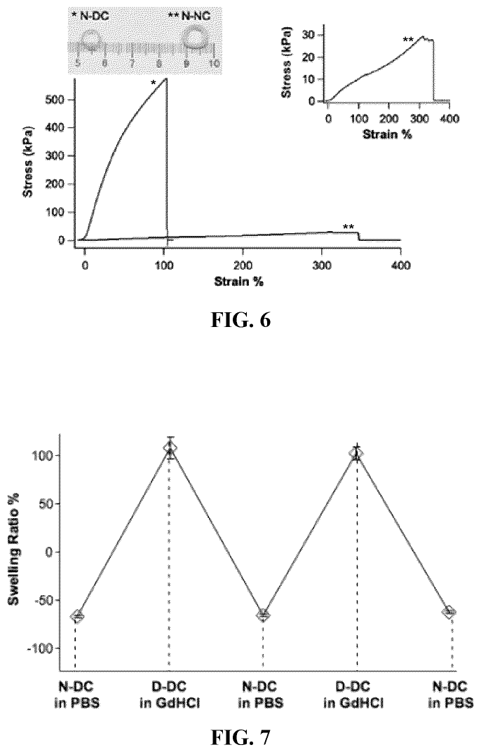

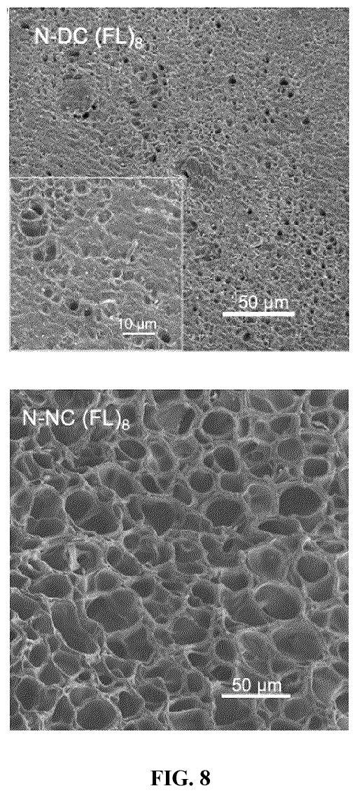

Chain entanglements in a chemically crosslinked network cannot be removed without breaking network strands. Thus, chain entanglements will be retained in the hydrogel network even if the polypeptide chains undergo conformational changes, such as folding. Taking advantage of this unique topological feature, the D-DC hydrogel was “renatured” in PBS to allow for refolding of FL domains. After reaching equilibrium, the native DC (N-DC) (FL)₈ hydrogel was obtained. The N-DC hydrogel deswelled dramatically compared with the D-DC hydrogel, and changed from being transparent to largely translucent (FIG. 6). In contrast, upon renaturation, the N-NC hydrogel deswelled from its D-NC form and became opaque. The swelling ratio of the N-DC hydrogel was smaller than that of the N-NC hydrogel (FIG. 6). Moreover, both DC and NC hydrogels can be cycled between their native and denatured states (N-DC to D-DC, N-NC to D-NC) reversibly for many cycles without noticeable change in their respective appearances and physical properties, such as swelling ratio (FIG. 7). While not wishing to be limited by theory, the deswelling of the N-DC hydrogel is likely due to the refolding of some FL domains in PBS, which is accompanied by a significant shortening of (FL)₈ (from 260 nm to 10 nm), and hydrophobic collapse of FL domains that remain unfolded (Fang & Li, 2012). Scanning electron microscopy imaging revealed that both N-DC and N-NC hydrogels showed microporous structures, but the pore size of N-DC hydrogel was significantly smaller than that of the N-NC hydrogel (FIG. 8), consistent with the smaller swelling ratio of the N-DC hydrogel. The pore size of the N-DC hydrogel is about 2 μm and the pore size of the N-NC hydrogel is about 20 μm.

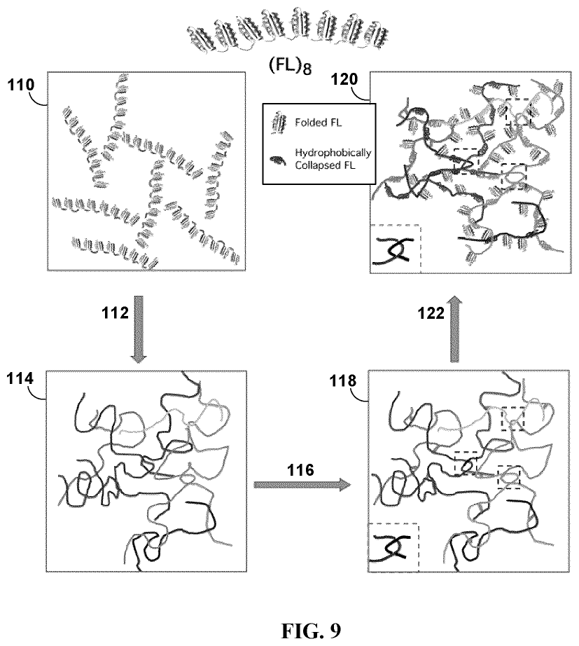

Due to the presence of chain entanglements, it was expected that in the N-DC gel, some FL domains would not be able to refold, instead, undergo hydrophobic collapse, as PBS is a poor solvent for the unfolded FL domain. Therefore, it is likely that the N-DC (FL)₈ hydrogel network assumed a single network structure consisting of both folded FL domains and unfolded ones, as schematically shown in FIG. 9. Referring to the schematics of the preparation of D-DC and N-DC (FL)₈ hydrogels in FIG. 9, the denatured crosslinked (DC) hydrogels were prepared by crosslinking unfolded proteins in their concentrated solutions. First, a concentrated solution of the native, folded elastomeric protein (FL)₈ 110 was prepared in PBS (with a concentration of 200 mg/mL). (FL)₈ was then denatured 112 using GdHCl and the unfolded (FL)₈ polypeptide chains, which behaved as random coils, likely overlapped with one another in the concentrated denatured protein solution 114, leading to possible chain entanglements. Upon photochemical crosslinking 116, the unfolded protein solution was crosslinked into the D-DC hydrogel 118, in which (FL); remain unfolded. After crosslinking, chain entanglements were retained in the hydrogel, leading to a network of entangled polypeptide chains. Entangled chains are highlighted in dashed squares. The lower left shows a zoomed view of one such chain entanglement. Referring to FIG. 9, the D-DC hydrogel 118 was converted to N-DC hydrogel 120 by renaturing 122 (FL) s in PBS. Upon renaturation, some FL domains refolded (as highlighted by the α/β structure of FL), while others underwent hydrophobic collapse (as indicated by irregular aggregate structure); see legend inset showing a schematic of the folded FL (upper) and a schematic of the hydrophobically collapsed FL (lower). In the N-DC hydrogel 120, the chain entanglements remained (highlighted in dashed squares; including a zoomed view of one such chain entanglement in the lower left), making the N-DC hydrogel 120 an entangled network of folded and hydrophobically collapsed proteins. To help visualize the entanglements, each individual (FL); molecule is shaded separately in 114, 118 and 120.

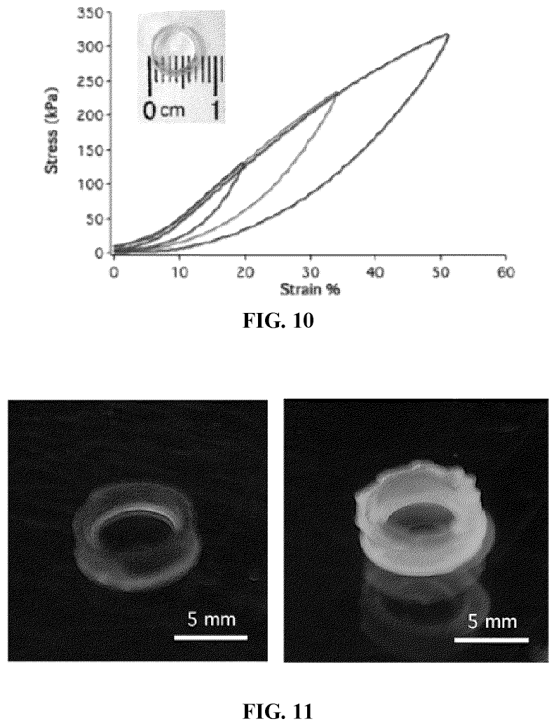

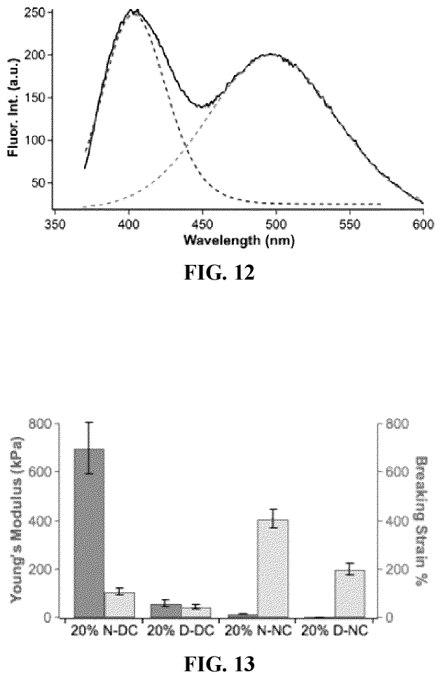

To verify the existence of unfolded FL domains in the N-DC hydrogel, a well-established cysteine shotgun labeling approach was used, which allows for labeling of only solvent-exposed cysteine residues using the thiol reactive fluorescent dye 5-((2-((iodoacetyl)amino)ethyl)amino)naphthalene-1-sulfonic acid (IAEDANS) (Johnson et al., 2007). For this experiment, a cysteine variant FL-M23C was used, in which the buried residue Met23 was mutated to Cys. Cys23 is sequestered in the hydrophobic core of the folded FL and can only be labeled with IAEDANS when FL-M23C is unfolded (Fang & Li, 2012). The N-DC (FL-M23C)₈ hydrogels showed similar physical and mechanical properties as (FL)₈ (FIG. 10). After IAEDANS labeling in PBS, the N-DC hydrogel showed the characteristic cyan fluorescence of IAEDANS under UV illumination (FIG. 11). Quantitative analysis showed that 50±6% (n=3) of the FL domains were unfolded in the N-DC hydrogel (FIG. 12), confirming that the N-DC hydrogel is a single network hydrogel consisting of folded and unfolded FL domains.

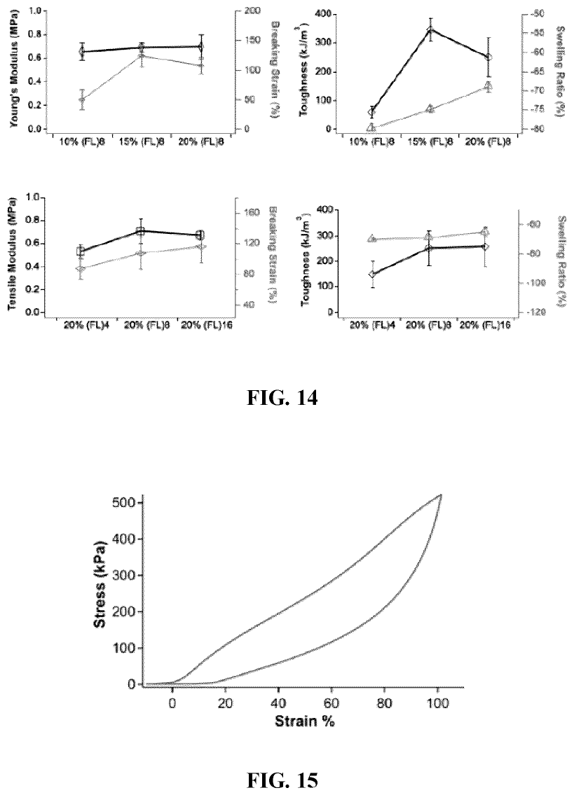

The tensile and compressive properties of the N-DC (FL)₈ hydrogel were then characterized. Typical stress-strain curves of 20% N-DC and N-NC hydrogels in PBS are shown in FIG. 6. It is evident that the N-DC hydrogel was much stiffer than the N-NC and D-DC hydrogels. The N-DC hydrogel displayed a Young's modulus E of 0.70±0.11 MPa (n=18) (FIG. 6 and FIG. 13), significantly higher than the Young's moduli of protein-based hydrogels reported in the literature (Lv et al., 2010; Wu et al., 2018; Elvin et al., 2005; McGann et al., 2013; Partlow et al., 2014). It was also significantly higher than that of the N-NC hydrogel (about 20 kPa). The breaking strain of the N-DC hydrogel was 107±14%, indicative of its good stretchability (FIG. 13). Similar Young's modulus and breaking strain were also observed for 15% N-DC (FL)₈ hydrogel (FIG. 14). However, 10% N-DC (FL)₈ hydrogel showed a similar Young's modulus but much smaller breaking strain (50%) (FIG. 14). Similar mechanical properties were also observed in 20% N-DC hydrogels based on (FL)₄ and (FL)₁₆, which have 4 and 16 tandem repeats of FL, respectively (FIG. 14), suggesting that the length of unfolded (FL)₄ (about 130 nm) is sufficient for chain entanglement to occur.

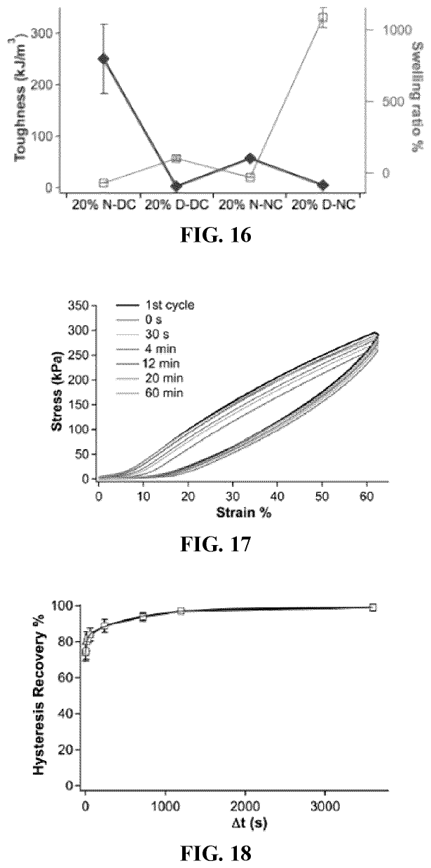

Moreover, the N-DC hydrogel exhibited a high tensile toughness (defined as the energy absorbed before fracturing, i.e. the area under the stress-strain curve) of 250±68 KJ/m³, and a large hysteresis was observed between the stretching-relaxation cycles, indicative of the large energy dissipation (FIG. 15 and FIG. 16). While not wishing to be limited by theory, the energy dissipation was likely due to the unfolding of a small number of FL domains, similar to what have been observed in protein hydrogels and muscle fibers (Lv et al., 2010; Lei et al., 2020; Minajeva et al., 2001). The N-DC (FL)₈ hydrogel also displayed fast recovery of its shape and mechanical properties (FIG. 17). After stretched to 60% strain and then relaxed to zero strain, 70% of the original energy dissipation capability recovered right after the hydrogel was relaxed, and the rest 30% recovered more slowly (FIG. 18). While not wishing to be limited by theory, this high energy dissipation and fast recovery are likely resulted from the forced-unfolding, and subsequent refolding of FL domains in the hydrogel network (Fang et al., 2013).

Evidently, the tensile properties of the N-DC (FL)₈ hydrogels are a unique combination of a high Young's modulus (about 0.7 MPa), high toughness and fast recovery, which are often in conflict to one another. Additionally, the high Young's modulus and toughness of this single network protein hydrogel are amongst the highest of the engineered protein hydrogels, and approaching the Young's moduli of articular cartilage as well as polymer hydrogels with unique network structures (Table 5).

TABLE 5
Mechanical properties of protein hydrogels and polymer hydrogels.

| | | | | |
|---|---|---|---|---|
| | Breaking | | | |
| Young's | stress | Breaking | | |
| modulus | (σₘₐₓ, | strain | | |
| (E, MPa) | MPa) | (εₘₐₓ) | Conc.* | Ref. |

Tensile

| Cartilage | Articular cartilage | | 1-10 | 1-20 | — | — | Kerin et al., |
|---|---|---|---|---|---|---|---|
| | | | | | | | 1998; |
| | | | | | | | Williamson |
| | | | | | | | et al., 2003; |
| | | | | | | | Wainwright |
| | | | | | | | et al., 1982. |
| N-DC | (FL)₈ | | 0.7 | 0.6 | 107% | 20% | — |
| Protein | GRG5RG4R | | 0.050 | 0.070 | 100% | 20% | Lv et al., |
| Hydrogels | | | | | | | 2010 |
| | (FL)₈ | | 0.016 | 0.035 | 450% | 20% | Fang et al., |
| | | | | | | | 2013 |
| | Bovine fibrinogen | | 0.070 | 0.046 | 62% | 20% | Elvin et al., |
| | | | | | | | 2009 |
| | Gelatin | | 0.070 | — | — | 10% | Gan et al., |
| | | | | | | | 2019 |
| | Gelatin | | 0.067 | 0.091 | 145% | 17.5% | Elvin et al., |
| | | | | | | | 2010 |
| | Regenerated silk fibroin | | 0.050 | — | — | 5% | Partlow et |
| | | | | | | | al., 2014 |
| | Gell crosslinked with | | 0.010-0.15 | 0.020 | 16% | 18% | Wu et al., |
| | 4-arm PEG | | | | | | 2018 |
| | (GB1)8-RX3 | | 0.065 | 0.11 | 80% | 20% | Fang & Li, |
| | | | | | | | 2012 |
| | Regenerated silk fibroin | | 2 | 0.7 | 110% | 10% | Su et al., |
| | (alcohol treated) | | | | | | 2017 |
| | Regenerated silk fibroin | | — | — | — | — | Numata et |
| | (alcohol treated double | | | | | | al., 2017 |
| | network) | | | | | | |
| Polymer | Double | PAMPS- | — | — | — | — | Gong et al., |
| hydrogels | network | PMAAm | | | | | 2003 |
| | | Bacterial | 21 | 3.8 | 28% | 50% | Nakayama |
| | | cellulose- | | | | | et al., 2004 |
| | | gelatin | | | | | |
| | | Collagen- | — | — | — | — | Gong et al., |
| | | PDMAAm | | | | | 2003 |
| | Cojoined | Chitosan- | 2.5 | 4.3 | 350% | 4% + 20% | Xu et al., |
| | network | gelatin- | | | | | 2019 |
| | | phytate | | | | | |
| | Slide-ring | NIPA- | 0.043 | 0.040 | 450% | 2M | Bin Imran |
| | network | AAcNa- | | | | (monomer) | et al., 2014 |
| | | HPR-C | | | | | |
| | Polymer- | ANF-PVA | 1.9-9.1 | 1.4-5.0 | 325% | 18-30% | Xu et al., |
| | composite | | | | | | 2019 |
| | Polymer- | g-CN- | 0.017 | — | — | 11% | Kumru et |
| | carbon | AHPA | | | | | al., 2019 |
| | nitride | | | | | | |
| | composite | | | | | | |
| | LP- | P(AAm(x)/ | — | 1.8 | 2000% | 27% | Ren et al., |
| | reinforced | HMA(y))- | | | | | 2017 |
| | | LPs(j) | | | | | |

Compression

| Cartilage | Articular cartilage | | 0.5-10 | 10-50 | — | — | Kerin et al., |
|---|---|---|---|---|---|---|---|
| | | | | | | | 1998; |
| | | | | | | | Williamson |
| | | | | | | | et al., 2003; |
| | | | | | | | Wainwright |
| | | | | | | | et al., 1982. |
| N-DC | (FL)₈ | | 1.7 | 68 | 82% | 20% | — |
| Protein | GRG5RG4R | | — | — | — | — | Lv et al., |
| Hydrogels | | | | | | | 2010 |
| | (FL)₈ | | — | — | — | — | Fang et al., |
| | | | | | | | 2013 |
| | Bovine fibrinogen | | — | — | — | — | Elvin et al., |
| | | | | | | | 2009 |
| | Gelatin | | 0.175 | 0.5 | — | 10% | Gan et al., |
| | | | | | | | 2019 |
| | Gelatin | | — | — | — | — | Elvin et al., |
| | | | | | | | 2010 |
| | Regenerated silk fibroin | | 0.020 | — | — | 5% | Partlow et |
| | | | | | | | al., 2014 |
| | Gell crosslinked with | | — | — | — | — | Wu et al., |
| | 4-arm PEG | | | | | | 2018 |
| | (GB1)8-RX3 | | — | — | — | — | Fang & Li, |
| | | | | | | | 2012 |
| | Regenerated silk fibroin | | 0.3-3 | 0.5-1.7 | about | 10% | Su et al., |
| | (alcohol treated) | | | | 60% | | 2017 |
| | Regenerated silk fibroin | | 5.8 | 12.4 | 80% | 25% | Numata et |
| | (alcohol treated double | | | | | | al., 2017 |
| | network) | | | | | | |
| Polymer | Double | PAMPS- | 0.33 | 17 | 92% | 10% | Gong et al., |
| hydrogels | network | PMAAm | | | | | 2003 |
| | | Bacterial | 3.9 | 5.3 | 44% | 50% | Nakayama |
| | | cellulose- | | | | | et al., 2004 |
| | | gelatin | | | | | |
| | | Collagen- | — | 2.9 | 53% | 13% | Gong et al., |
| | | PDMAAm | | | | | 2003 |
| | Cojoined | Chitosan- | 6.6 | 64 | 87% | — | Xu et al., |
| | network | gelatin- | | | | | 2019 |
| | | phytate | | | | | |
| | Slide-ring | NIPA- | — | — | — | — | Bin Imran |
| | network | AAcNa- | | | | | et al., 2014 |
| | | HPR-C | | | | | |
| | Polymer- | ANF-PVA | 1.4-4.0 | 5.9-26.5 | — | 18-30% | Xu et al., |
| | composite | | | | | | 2019 |
| | Polymer- | g-CN- | — | 10.8 | about | 11% | Kumru et |
| | carbon | AHPA | | | 60% | | al., 2019 |
| | nitride | | | | | | |
| | composite | | | | | | |
| | LP- | P(AAm(x)/ | — | 13.5 | 90% | 27% | Ren et al., |
| | reinforced | HMA(y))- | | | | | 2017 |
| | | LPs(j) | | | | | |

*polymer concentration in the as-prepared state.

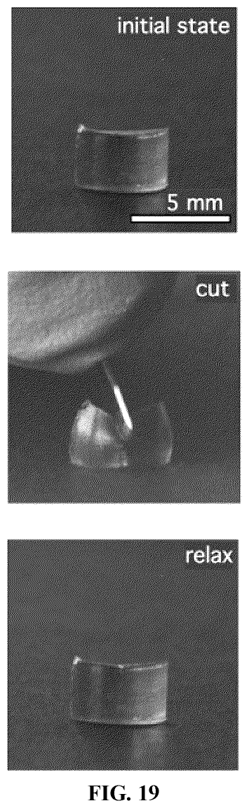

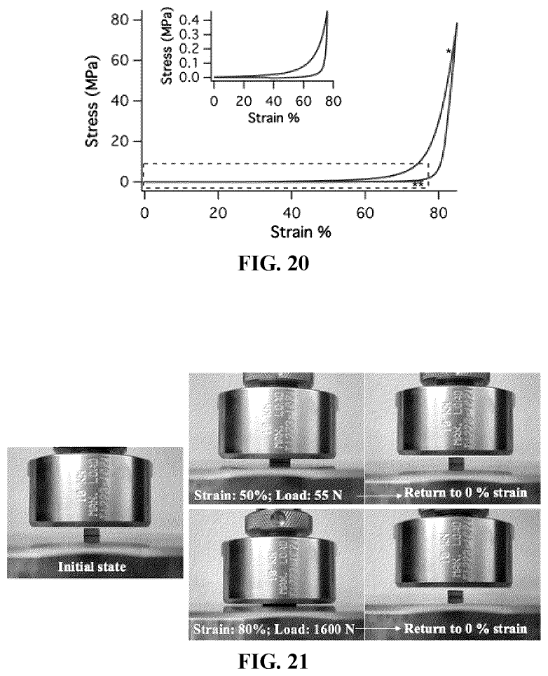

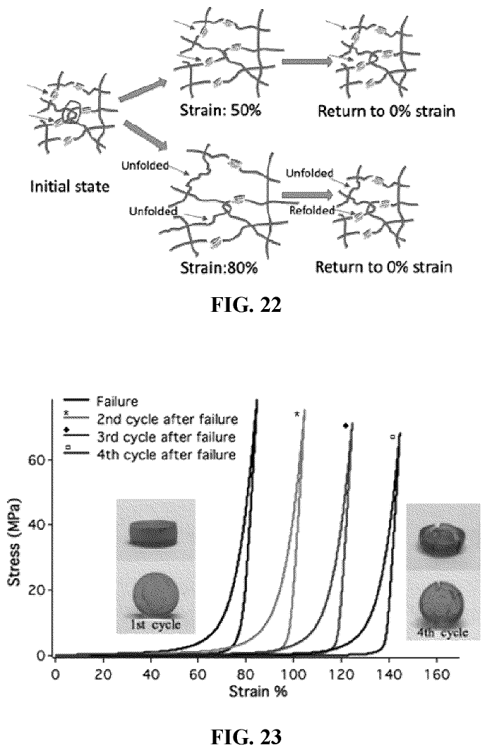

In addition to these excellent tensile properties, the N-DC (FL)₈ hydrogel demonstrated even more striking compressive properties. It was found that the N-DC (FL)₈ hydrogel is super tough and can resist slicing with a sharp scalpel, despite that it contains about 60% water (FIG. 19), indicating that the N-DC (FL)₈ hydrogel can efficiently dissipate the compression energy in the hydrogel network. To quantify their compressive mechanical properties, standard compression tests were carried out (FIG. 20). The stress-strain curves showed that the N-DC (FL)₈ hydrogels displayed a compressive modulus of about 1.7 MPa at 10-20% strain. In comparison, the N-NC (FL)₈ hydrogels only showed a compressive modulus of about 50 kPa (FIG. 20, inset), again revealing the significant enhancement effect of chain entanglements on the stiffness of the (FL)₈ hydrogels. As the strain increased, the stress of the N-DC (FL)₈ hydrogels increased more rapidly. Strikingly, the N-DC FL hydrogel could be compressed to more than 80% strain and sustain a compressive stress as high as 75 MPa without fracture (FIG. 21), suggestive of large energy dissipation during compression, which, while not wishing to be limited by theory, likely arose from the forced-unfolding of some FL domains in the network (FIG. 22). The structure is drawn in a highly schematic manner, and for illustration purpose only. The circle indicates the chain entanglement, arrows point to two folded FL domains that may undergo forced-unfolding at high strain, and then refolding after unloading. When compressed from the initial state (left) to 50% strain (top center) and then unloaded, the hydrogel can quickly recover (top right). When compressed to 80% strain (bottom center), a small number of FL domains unfold. Upon unloading, a large fraction of unfolded FL domains refolds quickly, while a small fraction refolds following a slower kinetics. For simplicity, hydrophobically collapsed FL aggregates are not shown.

The average compressive strength of the N-DC (FL)₈ hydrogel is 68±12 MPa (n=7), and an average failure strain of 82±3%. At the failure strain, a small crack often started to appear in the hydrogel. However, the failure was not brittle, as evidenced by the subsequent stress-strain curves of the same hydrogel sample (FIG. 23). These results demonstrated the ultrahigh compressive modulus, strength and toughness of the N-DC (FL)₈ hydrogel. The compressive modulus (1.7 MPa) and strength (68 MPa) of the N-DC (FL)₈ hydrogel are amongst the highest achieved by hydrogels (Table 5), and compare favorably with that of articular cartilage (0.2-10 MPa in modulus and 10-50 MPa in strength) (Hayes & Mockros, 1971; Kerin et al., 1998; Lu et al., 2008). As a comparison, the super tough double-network polymer hydrogels showed a compressive modulus of 0.3-3.9 MPa and fractured at a stress of no more than 20 MPa (Gong et al., 2003; Gong et al., 2010) the poly(vinyl alcohol)/nanofiber composite hydrogel has a modulus of 1-4 MPa and a fracture strength of 6-26 MPa (Xu et al., 2018), and co-joined network Chitosan-gelatin phytate hydrogel had a compressive modulus of 6.6 MPa and strength of 64 MPa (Xu et al., 2019).

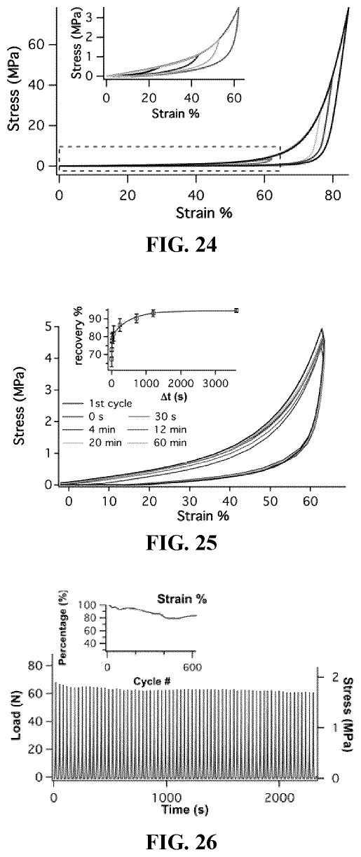

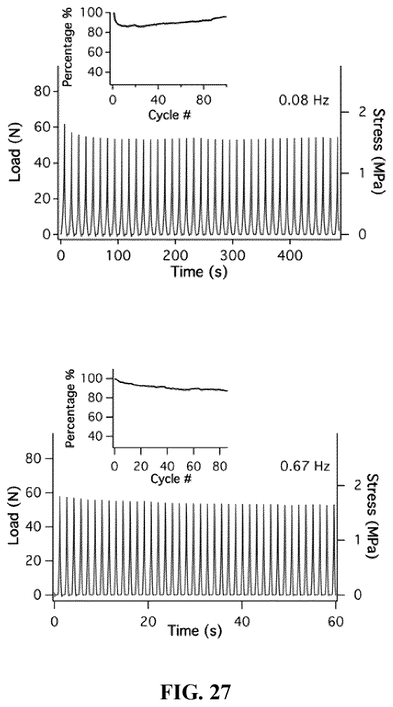

In the compression-unloading cycles, a large hysteresis was observed (FIG. 24), indicative of a large energy dissipation. The dissipated energy increased as the strain increased. At 80% strain, the hydrogel exhibited a toughness of 3.2±0.6 MJ/m³ (n=7). Moreover, the N-DC hydrogel displayed excellent recovery properties (FIG. 25). At lower strains (<40%), the hydrogel recovered its dimension right at the end of each loading-unloading cycle. Even at larger strains (>60%), about 65% of the original energy dissipation capability recovered right after unloading, and the remaining 35% recovered within an hour (FIG. 25). Furthermore, the hydrogel did not show much fatigue on a time scale of 2 hr after 600 consecutive loading-unloading cycles at a frequency of up to 0.67 Hz and a final strain of 60% (FIG. 26 and FIG. 27).

Collectively, these results revealed that the N-DC (FL)₈ hydrogels are mechanically stiff and tough, and can recover their shape and mechanical properties rapidly and do not show much mechanical fatigue. Moreover, these protein hydrogels showed excellent long-term stability: after stored in PBS (with 0.2% % NaN₃) for over eight months, their physical shape and mechanical properties remained largely unchanged. These exceptional mechanical properties and their unique integration in one material are rare for protein hydrogels, and compare favorably with those of polymer hydrogels with special network structure (Table 5). These properties closely reproduced many mechanical features of articular cartilage, making the N-DC (FL)₈ hydrogel a biomaterial that mimic the mechanical properties of articular cartilage. It is important to note that other than biomechanical mimicry, this material does not mimic or capture other properties of cartilage, such as spatial structuring and transport properties.

While not wishing to be limited by theory, these outstanding mechanical properties of N-DC (FL)₈ hydrogels likely result from a combination of factors, including chain entanglement, a hybrid hydrogel network which includes both folded and hydrophobically collapsed FL domains, as well as the forced unfolding and refolding of FL domains. Amongst these factors, chain entanglements in the folded protein hydrogel network play an essential role, owing to their ability to enhance the stiffness of the hydrogels without compromising toughness. Different from chemical crosslinks, chain entanglements are “mobile” in the network. This unique feature prevents hydrogels from getting brittle while being stiffened, a feat that cannot be achieved by simply increasing chemical crosslinking density. Since the presence of chain entanglements are general in concentrated unfolded protein solution (and polymer solution), this DC method for protein hydrogelation should be broadly applicable. Indeed, as shown in Table 6, based on this DC method, N-DC protein hydrogels were engineered using a variety of elastomeric proteins, ranging from all a proteins to α/β proteins, which all exhibited significantly enhanced stiffness over their N-NC counter parts. Similar enhancement was also achieved in their compressive moduli (Table 6).

TABLE 6
Enhancement of mechanical properties via the DC hydrogelation method

| | Tensile | | | Compression | | |
|---|---|---|---|---|---|---|
| | N-DC | N-NC | | N-DC | N-NC | |
| | hydrogel | hydrogel | | hydrogel | hydrogel | |
| | E_{N-DC} | E_{N-NC} | Enhancement | Y_{N-DC} | Y_{N-NC} | Enhancement |
| Protein | (kPa) | (kPa) | E_{N-DC}/E_{N-NC} | (kPa) | (kPa) | Y_{N-DC}/Y_{N-NC} |
| (FL)₈ | 700 ± 110 | 15.7 ± 1.8 | 44 | 1614 ± 201 | 40.1 ± 2.7 | 40 |
| (NuG2)₈ | 620 ± 93 | 11.2 ± 3.3 | 56 | 1086 ± 31 | 55.0 ± 11 | 20 |
| NRN₄RN₄R | 126 ± 21 | 22.1 ± 2.7 | 5.7 | 592 ± 148 | 114.0 ± 25.1 | 5.2 |
| (GB1)₈ | 227 ± 30 | 4.8 ± 1.1 | 47 | 339 ± 36 | 68.3 ± 10.5 | 5.0 |
| GRG₅RG₄R | 123 ± 53 | 32.7 ± 4.2 | 3.8 | 220 ± 31 | 19.6 ± 3.9 | 11 |
| BSA | 236 ± 5 | 6.5 ± 0.1 | 36 | 776 ± 173 | 19.5 ± 3.9 | 40 |
| (GA)₈ | 438 ± 55 | 60.1 ± 9.0 | 7.3 | 1704 ± 248 | 95.0 ± 17 | 18 |
| (FL-M23C)₈-Fn | 380 ± 76 | — | — | 834 ± 280 | — | — |
| FLRGD | 179 ± 8 | — | — | 206 ± 15 | — | — |

In Table 6, E is tensile modulus; Y is compressive modulus; (NuG2)₈ is a polyprotein made of eight tandem repeats of the protein NuG2 (Cao et al., 2008); (GB1)₈ is a polyprotein made of eight tandem repeats of the protein GB1 (Cao et al., 2007); in GRG₅RG₄R, G represents GB1 domain, and R represents the 15 residue consensus sequence of resilin (Lv et al., 2010); in NRN4RN4R, N represents NuG2 domain, and R represents the 15 residue consensus sequence of resilin; BSA is bovine serum albumin (Khoury et al., 2019); (GA)₈ is a polyprotein made of eight tandem repeats of the protein GA (Alexander et al., 2009); FLRGD is Fn-(Cys-FL)₄-RGD-(Cys-FL)₄-RGD, in which RGD represents the 17 residue sequence derived from fibronectin (Leahy et al., 1996).

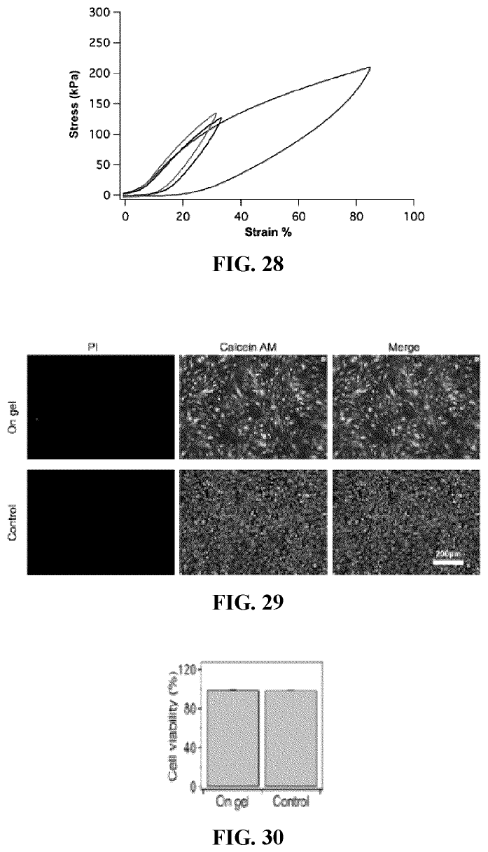

In addition, this method can be readily adapted to other crosslinking chemistry. For example, using disulfide crosslinking (oxidized by either air oxygen or H₂O₂), N-DC (FL-M23C)₈ hydrogels were successfully prepared, whose mechanical properties (FIG. 28) are comparable to those of (FL)₈ hydrogels prepared via photochemical crosslinking.

It is important to stress that chain entanglements and a hybrid protein network comprising both folded and hydrophobically collapsed proteins are required to achieve the high stiffness and high toughness for N-DC hydrogels. Missing either one will lead to the loss of high stiffness or both high stiffness and toughness. As detailed above, the possible mechanism underlying the high stiffness and toughness of N-DC hydrogels likely results from chain entanglements and a hybrid protein network comprising both folded and hydrophobically collapsed proteins. Proteins that are intrinsically disordered or lack a folded globular structure are not suitable building blocks for engineering stiff and tough protein hydrogels using the DC hydrogelation method. For example, since gelatin does not have a folded globular structure in its folded state, the N-DC gelatin hydrogels are soft (with a Young's modulus of 10 kPa), similar to the N-NC gelatin hydrogels. The possible structural changes of the N-DC (FL)₈ hydrogel under compression are shown in FIG. 22. The force-induced unfolding of the folded FL domains help dissipate energy during loading, and upon unloading, the refolding of FL domains help the hydrogel to regain its shape and mechanical properties. In addition, it appears that the globular protein itself and the presence of intrinsically disordered protein sequences (such as resilin) may modulate the stiffness and toughness of the engineered N-DC hydrogels, thus enabling fine-tuning the mechanical properties of the engineered stiff N-DC hydrogels. Regarding modulation of the stiffness of the N-DC hydrogels of the resultant N-DC hydrogels, two empirical experimental approaches have been identified to tune the mechanical properties of the N-DC hydrogels. The first one is to choose different folded globular proteins as building blocks to construct elastomeric proteins. Different proteins will have different mechanical stability in their folded state, and different tendency to undergo hydrophobic collapse in their unfolded state in PBS. These differences will affect the stiffness and toughness (as shown in Table 6). The second one is to incorporate intrinsically disordered protein sequences (such as resilin) in the elastomeric proteins. The incorporation of an unstructured protein sequence between folded globular domains will increase the flexibility of the elastomeric protein. The increased flexibility appears to soften the N-DC hydrogel network, leading to reduced stiffness. For example, incorporating resilin or RGD sequence in the elastomeric proteins led to the reduction of the stiffness of the corresponding N-DC hydrogels (Table 6). The detailed molecular mechanisms underlying these empirical approaches remain to be elucidated. Nonetheless, these approaches offer the possibility to fine-tune the high stiffness of N-DC protein hydrogels for different applications. As shown in Table 6, by using different globular proteins and/or incorporating disordered protein sequences into tandem modular proteins, it is possible to tune the mechanical properties of stiff protein hydrogels, achieving Young's modulus from, for example 0.18 MPa to 0.70 MPa, and compressive modulus from 0.21 MPa to 1.7 MPa. These results demonstrate the adaptability of this method towards engineering stiff and tough protein-based biomaterials.

Proteins are attractive building blocks to construct biomaterials, but protein hydrogels are generally soft and inept to mimic stiff tissues (Table 5). A DC hydrogelation approach was demonstrated to enable the engineering of stiff and tough protein hydrogels. The key of this approach is to introducing chain entanglements into the folded protein network to resolve the incompatibility between stiffness and toughness, that is to stiffen but not embrittle the protein hydrogel network. On the one hand, chain entanglement allows the hydrogel to achieve high stiffness. On the other hand, forced-unfolding of globular proteins provides an efficient mechanism for energy dissipation, and the ability to refold allows the hydrogel to recovery its mechanical properties rapidly and minimize mechanical fatigue. In so doing, these effects work cooperatively to allow the integration of high stiffness, high toughness, fast recovery and high compressive strength into protein hydrogels. These results demonstrate that it is now possible to use the NC and DC hydrogelation methods to use the same elastomeric protein building blocks to engineer both soft protein biomaterials, whose stiffness is close to that of muscle, as well as stiff biomaterials, whose stiffness is close to that of cartilage, thus significantly expanding the range of mechanical properties that protein hydrogels can achieve. To some extent, using the DC hydrogelation method it has become possible to convert soft protein hydrogels into stiff biomaterials, effectively making the large number of existing protein hydrogels as potential candidates for developing highly stiff and tough protein hydrogels. The range of stiffness that N-DC protein hydrogels have achieved (Table 6) compares favorably with that of double network hydrogels (Table 5), yet the N-DC hydrogels comprise only a single network structure and are easy to construct. Given the generality of this approach, the richness of potential protein building blocks and the range of mechanical properties that can be achieved, this may thus open up an exciting new areas for further systematic exploration. As a result, the mechanical properties offered by these protein hydrogels now open up the possibility of using such stiff and tough protein hydrogels for applications that are not possible to achieve using soft protein hydrogels in different fields, ranging from biomedical engineering to material science and engineering. To test such possibilities, the utility of the stiff and tough protein hydrogels was explored in the repair of osteochondral defect.

Engineering soft and stiff FLRGD hydrogels for the repair of osteochondral defect: Given the mechanical properties of the N-DC hydrogels, the potential of such hydrogels as biomechanically compatible scaffolds for the repair of osteochondral defect was tested. For this, N-DC Fn-(Cys-FL)₄-RGD-(Cys-FL)₄-RGD hydrogel (for convenience, it is referred to as FLRGD) was engineered, where Fn is the third FnIII domain of human extracellular matrix protein tenascin (Leahy et al., 1992) and RGD is a 17 aa residue long sequence, TVYAVTGRGDSPASSRS (SEQ ID NO:3) derived from cell adhesion protein fibronectin that contains the integrin-binding RGD (Arginine-Glycine-Glutamate) motif, both of which entail cell adhesion ability to the protein. To properly evaluate the role of hydrogel stiffness on the efficacy on repairing the osteochondral defect, both stiff N-DC and soft N-NC hydrogels are needed. To avoid any unknown adverse biological effects of residual ammonium persulfate (APS) and [Ru(bpy)₃]²⁺ used in the photochemical crosslinking reaction, the disulfide crosslinking method was used to engineer the N-DC FLRGD hydrogel. Since the cysteine residues in (FL-M23C)₈ are buried in the hydrophobic core in the folded state, it is not possible to engineer N-NC (FL-M23C)₈ hydrogels using disulfide-based crosslinking. In order overcome this hurdle, FLRGD (Fn-(Cys-FL)₄-RGD-(Cys-FL)₄-RGD) was engineered, in which Cys was placed between two neighboring FL domains. The N-DC FLRGD hydrogel can be readily crosslinked by air or H₂O₂ oxidation. The resultant N-DC hydrogel showed a compressive modulus Y of 0.21 MPa, which is at the lower end of the range of reported modulus of cartilage (Almarza & Athanasiou, 2004). However, it was not possible to oxidize folded FLRGD into a hydrogel, while not wishing to be limited by theory, possibly due to the steric hindrance of the folded protein structure that significantly limited the accessibility of Cys. Hence, a different crosslinking method was used to prepare the soft N-NC FLRGD hydrogel. A mixed aqueous solution of FLRGD and gelatin (5% FLRGD and 5% gelatin) at 40° C. was first prepared, and the solution allowed to cool so that gelatin can physically crosslink into a physical hydrogel. Then the physical gel was chemically crosslinked by using the NHS-EDC chemistry to obtain the N-NC FLRGD/gelatin hydrogel, which showed a compression modulus of 35 kPa. The NHS-EDC chemistry could not be used to prepare homogenous DC-FLRGD hydrogels, as the NHS-EDC chemistry-mediated crosslinking reaction proceeded too rapidly in solution. Thus, the stiff N-DC FLRGD hydrogel (with a compression modulus of 0.21 MPa) and the soft N-NC FLRGD/gelatin hydrogel serve as a pair of hydrogels with similar chemical compositions but significantly contrasting stiffness for evaluating the effect of stiffness on the repair of osteochondral defect.

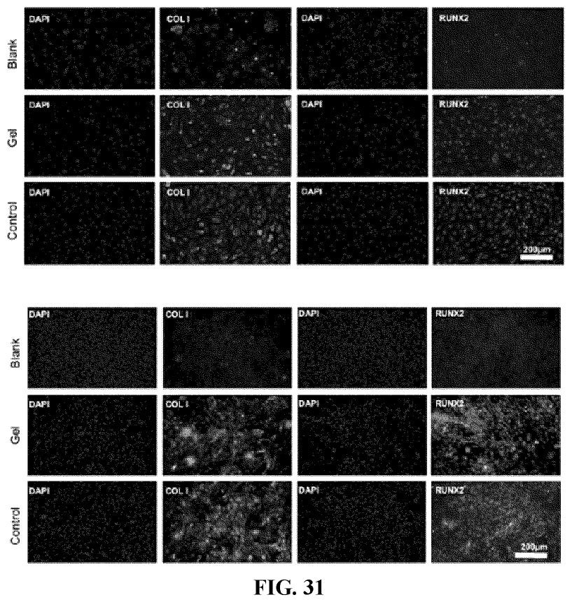

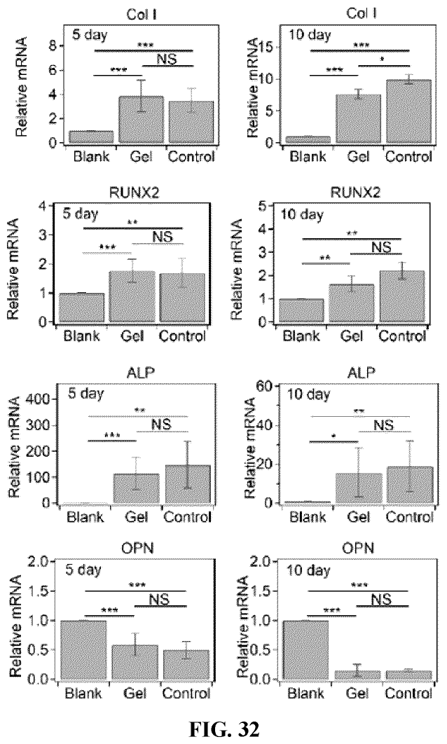

The biocompatibility was first characterized in vitro. In vitro experiments showed that the N-DC FLRGD hydrogel is fully biocompatible, and supports cell adhesion, spreading and proliferation (FIG. 29 and FIG. 30). In addition, mouse osteoprogenitor MC3T3-E1 cells cultured on the N-DC FLRGD hydrogels can differentiate into osteoblasts, as revealed by immunofluorescence staining and quantitative reverse transcription PCR (qRT-PCR) analysis (FIG. 31 and FIG. 32). Clearly, Col I and Runx2 were up-regulated in MC3T3-E1 cells cultured on N-DC FLGRD hydrogel (Gel) and on coated cell culture dish (control) groups, while Col I and Runx2 were hardly observed in blank (uncoated cell culture dish) groups (FIG. 31). This result indicated that N-DC FLRGD hydrogels provided a stiff, osteogenic matrix for MC3T3-E1, consistent with the mechanobiological findings that stiff matrix (with a modulus greater than 25 kPa) promotes osteogenic differentiation (Engler et al., 2006).

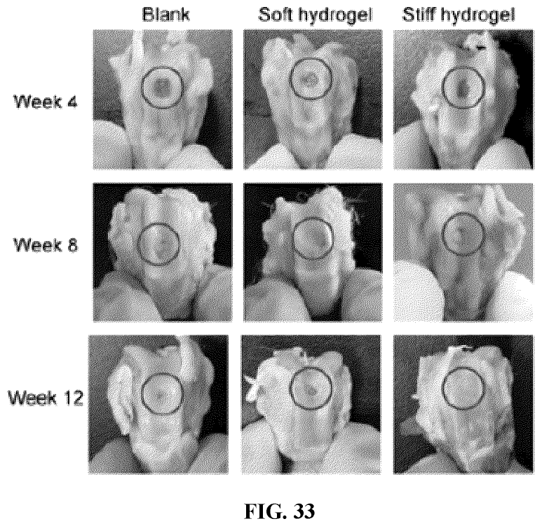

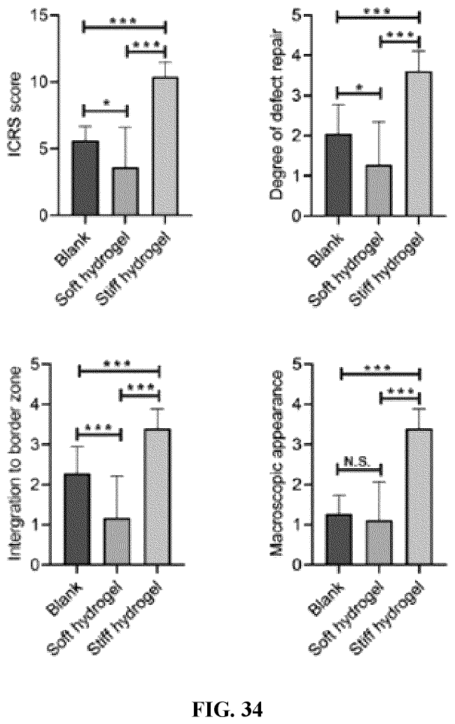

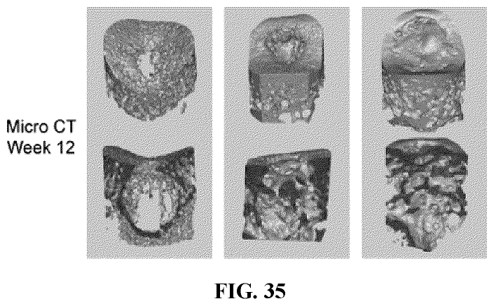

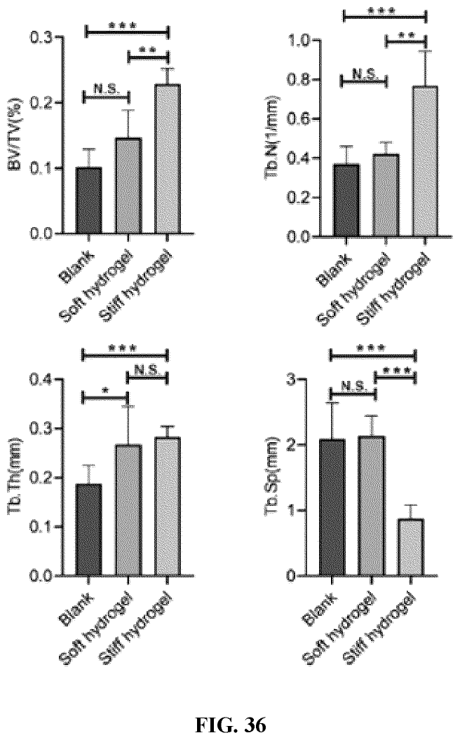

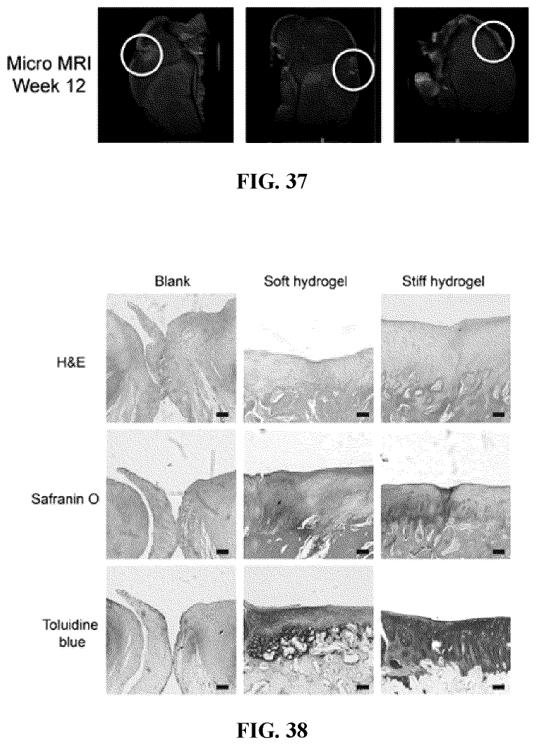

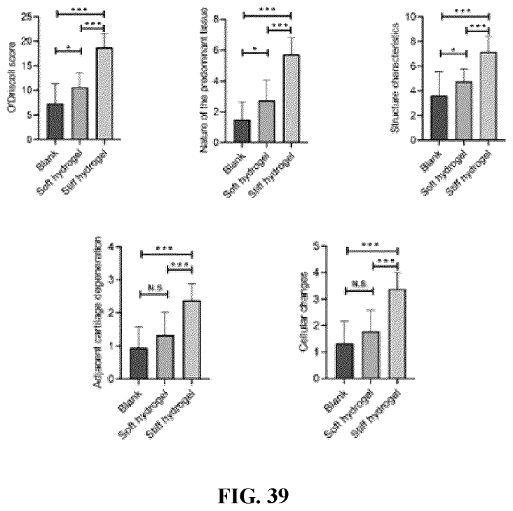

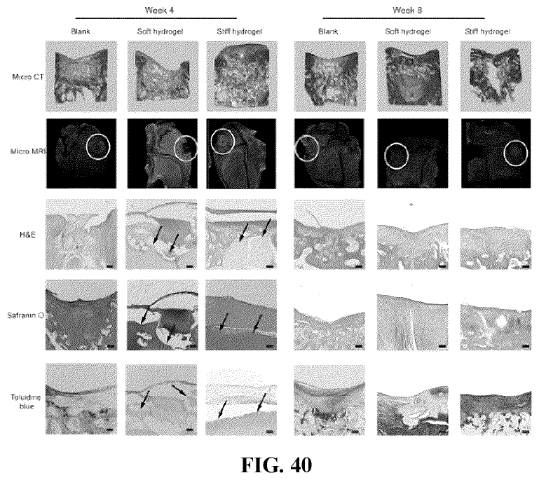

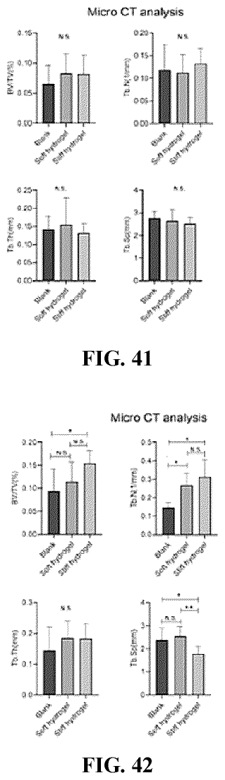

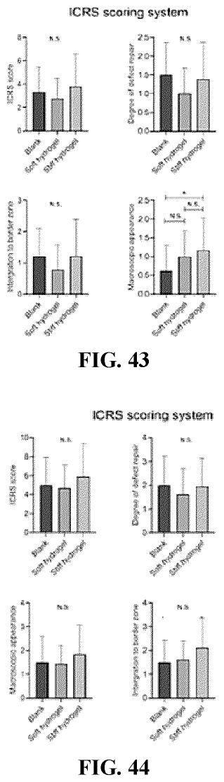

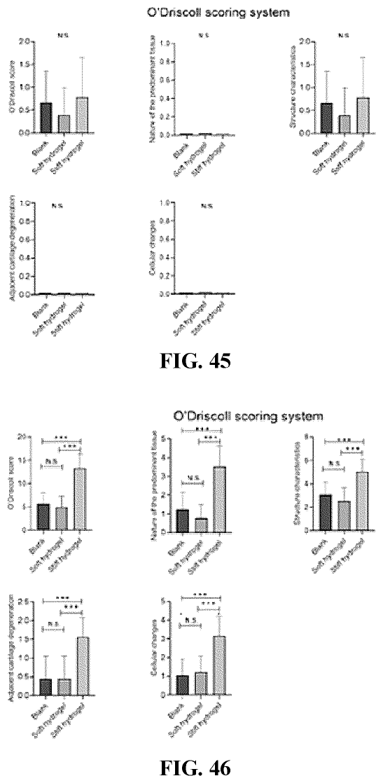

Building upon these successful in vitro experiments, the use of the N-DC FLRGD hydrogels as scaffolds for osteochondral defect repair in a rabbit model was then tested. To evaluate the effect of hydrogel stiffness of the N-DC hydrogels on the repair efficacy of osteochondral defect, “naked” hydrogels that only contain cell adhesive RGD motif but do not incorporate any growth factor were used: soft N-NC FLRGD/gelatin hydrogel (with a compressive modulus of 35 kPa), stiff N-DC FLRGD hydrogel (with compressive modulus of 0.21 MPa), and a blank as control. The repair of subchondral defect was evaluated after 4, 8 and 12 weeks implantation. The osteochondral defects were notably repaired in the stiff FLRGD hydrogels group after 12 weeks implantation (FIGS. 33-37), but not in the control and soft FLRGD/gelatin hydrogels. After 12 weeks implantation, no hydrogel was left in the defects in the soft and stiff hydrogel groups. The defects in the blank and soft hydrogel groups were filled with irregular and depressive regenerated tissues (FIG. 33), which were clearly distinguishable from the surrounding cartilage. In contrast, the regenerated tissues in the stiff FLRGD hydrogel group were covered by glossy and smooth membrane, which were close to the native cartilage (FIG. 33). The International Cartilage Repair Society (ICRS) scores also confirmed these results (FIG. 34). Micro computerized tomography (CT) analysis revealed the success in the repair of the subchondral bone in the stiff FLRGD hydrogel group (FIG. 35). Obvious newly-born bony tissues were observed in the stiff FLRGD hydrogel group, and the structure of the regenerated bone was similar to the surrounding tissue. In contrast, a cavity existed in the defect region in the blank and soft hydrogel groups. The stiff FLRGD hydrogel group exhibited higher Bone volume/Total volume (BV/TV), Trabecular Number Tb.N, and Trabecular Thickness (Tb.Th) (FIG. 36), indicating that significant osteogenesis occurred at the hydrogel treated region. In contrast, the control group had higher Trabecular Separation (Tb.Sp), indicative of notable bone resorption. Micro magnetic resonance imaging (MRI) analysis revealed similar trends (FIG. 37). Histological results (FIG. 38 and FIG. 39) showed that the defect of bone and cartilage in the stiff hydrogel group were filled by the regenerated tissue that were uniform and smooth and showed vertical arrangement of chondrocytes as native cartilage. In contrast, an obvious gap and cavity were observed in the blank and soft hydrogel groups. The extracellular matrix of the regenerated region showed clear regeneration of hyaline cartilage and active production of glycosaminoglycan (FIG. 38). Overall, the stiff FLRGD hydrogel group showed higher scores in total O'Driscoll evaluation and all the detail items (FIG. 39). Moreover, the degree of repair in the stiff FLRGD hydrogel group showed a clear progress with time, while blank control and soft hydrogel group did not (FIGS. 40-46). This result is consistent with the time course of tissue remodeling and regeneration.

Given that the soft and stiff FLRGD hydrogels have similar chemical composition but different stiffness, the superior repair efficacy achieved using the stiff N-DC FLRGD hydrogel can be reasonably attributed to the higher stiffness of the N-DC FLRGD hydrogels. It is likely that the higher stiffness, which is biomechanically more compatible with osteochondral bone and cartilage tissues, likely provides a suitable physical cue that is required for the effective regeneration of osteochondral bone and cartilage tissues. Although the detailed regeneration mechanism is unknown, while not wishing to be limited by theory, it is likely that the regeneration involves the mesenchymal stem cells released from osteochondral bone marrow (Huey et al., 2012). Regarding the possible mechanism for cartilage regeneration, cartilage regeneration is a complex process involving many factors (Huey et al., 2012). The detailed molecular mechanism underlying the cartilage regeneration promoted by the N-DC FLRGD hydrogel implants is yet to be understood. However, while not wishing to be limited by theory, it is likely that it follows a similar marrow stimulation mechanism on which the microfracture and augmented microfracture strategies are based (Huey et al., 2012). Through microfracture, which involves subchondral bone penetration, stem cells can be released from bone marrow and form a stem cell-rich clot. Hydrogel scaffold can then help promote stem cell's adhesion, proliferate and differentiation, leading to the regeneration of bone and cartilage tissues (Huey et al., 2012; Stanish et al., 2013). Systematic work can be carried out to elucidate the mechanistic details regarding the repair by the N-DC FLRGD hydrogel.

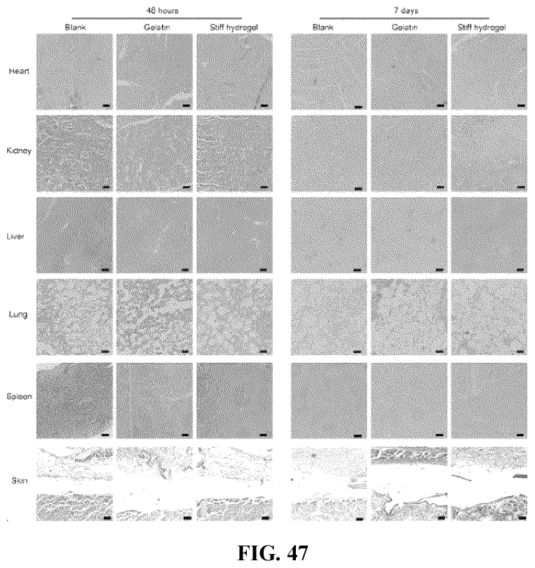

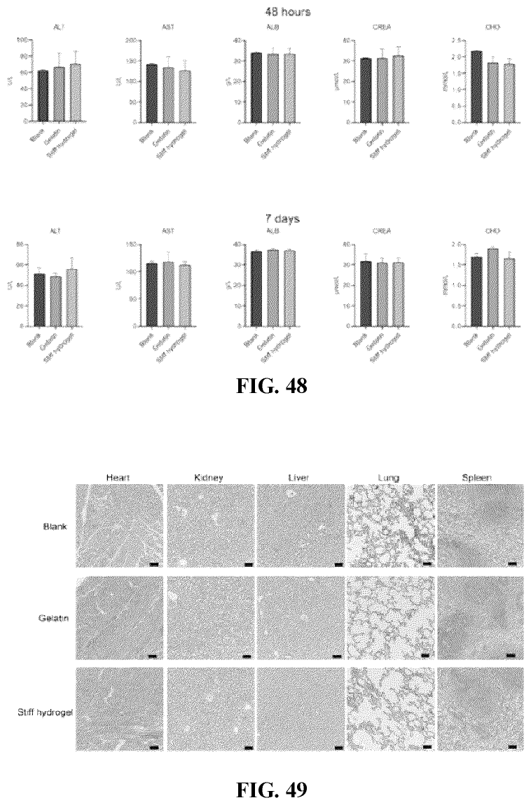

The results of an evaluation of immunological response of animals after hydrogel implantation are shown in FIGS. 47-49. After implantation for two and seven days, no obvious change can be seen in the major organs, including heart, liver, spleen, lung, and kidney (FIG. 47). The implanted region showed a similar inflammatory reaction to the blank group and no differences were found in liver function, including, as shown in FIG. 48: alanine transaminase (ALT), aspartate transaminase (AST), albumin (ALB), creatinine (CREA) and cholesterol (CHO). In addition, no pathological changes were found at the major organs of the rabbits at all three time points of 4, 8 and 12 weeks (FIG. 49, for simplicity, only the results at the time point of 12 weeks are shown). In sum, it is worth noting that the N-DC FLRGD hydrogel showed excellent in vivo biocompatibility, no immunological rejection, evaluated after 2 and 7 days, (FIGS. 47-48), or synovial proliferation, or pathological organ changes were observed in animals (FIG. 49). These results clearly demonstrate the excellent biocompatibility of the stiff FLRGD hydrogel.

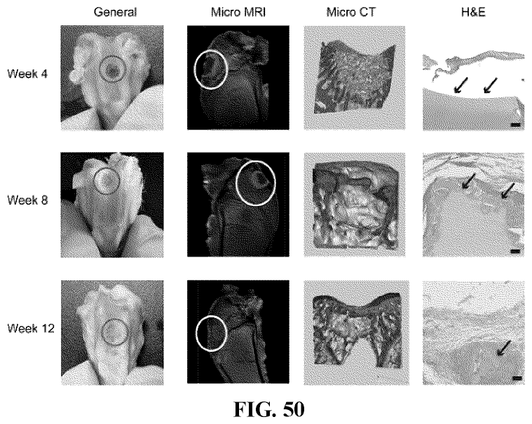

The efficacy of the highly stiff (FL-M23C)₈-Fn hydrogels (with a compressive modulus of 0.83 MPa) was also tested for the repair of osteochondral defect. Despite that its stiffness is even higher, highly stiff (FL-M23C)₈-Fn hydrogel did not lead to good repair of osteochondral defect (FIG. 50): only irregular and depressive tissues were regenerated (FIG. 50, left two columns), which were clearly distinguishable from the surrounding cartilage; and limited regeneration of subchondral bone tissues was observed in animals but a cavity remained in the defect region after 12 weeks implantation (FIG. 50, second column from the right). This outcome likely resulted from the much slower degradation kinetics of the highly stiff (FL-M23C)₈-Fn hydrogel in vivo. After 8 weeks implantation, significant amounts of hydrogels remained (FIG. 50, far right column). Even after 12 weeks, hydrogel remains were still observed in animals (FIG. 50, far right column). This much slower degradation profile likely led to a mis-match between tissue regeneration and scaffold degradation. This result highlights the complexity of cartilage regeneration, as well as the necessity in optimizing biomechanical cues with other biochemical and biophysical factors simultaneously for the design of hydrogel scaffolds for successful repair of osteochondral defect.

Taken together, these results suggest that hydrogel scaffolds with a suitable high stiffness enhances biomechanical compatibility of the hydrogel scaffold and improves the repair efficacy of osteochondral defect. These promising results on the stiff FLRGD hydrogels and the novel DC hydrogelation method now open an avenue to engineering protein hydrogels that will combine biomechanical cues with biochemical ones (such as growth factors) for their applications in the repair of osteochondral defect.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR REFERENCES REFERRED TO IN THE DESCRIPTION

Alexander, P. A., He, Y., Chen, Y., Orban, J. & Bryan, P. N. A minimal sequence code for switching protein structure and function. Proc Natl Acad Sci USA 106, 21149-21154, (2009).
Almarza, A. J. & Athanasiou, K. A. Design characteristics for the tissue engineering of cartilaginous tissues. Ann Biomed Eng 32, 2-17, (2004).
Bin Imran, A. et al. Extremely stretchable thermosensitive hydrogels by introducing slide-ring polyrotaxane cross-linkers and ionic groups into the polymer network. Nat Commun 5, 5124, (2014).
Cao, Y. & Li, H. Polyprotein of GB1 is an ideal artificial elastomeric protein. Nat Mater 6, 109-114, (2007).
Cao, Y., Kuske, R. & Li, H. Direct observation of markovian behavior of the mechanical unfolding of individual proteins. Biophys J 95, 782-788, (2008).
Colby, R. H. Structure and linear viscoelasticity of flexible polymer solutions: comparison of polyelectrolyte and neutral polymer solutions. Rhol. Acta 49, 425-442, (2010).
Discher, D. E., Janmey, P. & Wang, Y. L. Tissue cells feel and respond to the stiffness of their substrate. Science 310, 1139-1143, (2005).
Elvin, C. M. et al. Synthesis and properties of crosslinked recombinant pro-resilin. Nature 437, 999-1002, (2005).
Elvin, C. M. et al. The development of photochemically crosslinked native fibrinogen as a rapidly formed and mechanically strong surgical tissue sealant. Biomaterials 30, 2059-2065, (2009).
Elvin, C. M. et al. A highly elastic tissue sealant based on photopolymerised gelatin. Biomaterials 31, 8323-8331, (2010).
Engler, A. J., Sen, S., Sweeney, H. L. & Discher, D. E. Matrix Elasticity Directs Stem Cell Lineage Specification. Cell, (2006).
Fancy, D. A. & Kodadek, T. Chemistry for the analysis of protein-protein interactions: Rapid and efficient cross-linking triggered by long wavelength light. Chemistry Biochemistry, (1999).
Fang, J. et al. Forced protein unfolding leads to highly elastic and tough protein hydrogels. Nat Commun 4, 2974, (2013).
Fang, J. & Li, H. A facile way to tune mechanical properties of artificial elastomeric proteins-based hydrogels. Langmuir 28, 8260-8265, (2012).
Gan, D. et al. Mussel-Inspired Tough Hydrogel with In Situ Nanohydroxyapatite Mineralization for Osteochondral Defect Repair. Adv Healthc Mater 8, e1901103, (2019).
Gong, J. P., Katsuyama, Y., Kurokawa, T., Osada, Y. Double-network hydrogels with extremely high mechanical strength. Adv Mater 15, 1155-1158, (2003).
Gong, J. P. Why are double network hydrogels so tough? Soft Matter 6, 2583-2590, (2010).
Gosline, J. et al. Elastic proteins: biological roles and mechanical properties. Philosophical Transactions of the Royal Society of London Series B-Biological Sciences 357, 121-132, (2002).
Hayami, T. et al. The role of subchondral bone remodeling in osteoarthritis: reduction of cartilage degeneration and prevention of osteophyte formation by alendronate in the rat anterior cruciate ligament transection model. Arthritis Rheum 50, 1193-1206, (2004).
Hayes, W. C. & Mockros, L. F. Viscoelastic properties of human articular cartilage. Journal of applied physiology, (1971).
Higuchi, H. Viscoelasticity and function of connectin/titin filaments in skinned muscle fibers. Adv Biophys 33, 159-171, (1996).
Huey, D. J., Hu, J. C. & Athanasiou, K. A. Unlike bone, cartilage regeneration remains elusive. Science 338, 917-921, (2012).
Hunziker, E. B. Articular cartilage repair: basic science and clinical progress. A review of the current status and prospects. Osteoarthritis Cartilage 10, 432-463, (2002).
Jiang, Y. & Tuan, R. S. Origin and function of cartilage stem/progenitor cells in osteoarthritis. Nat Rev Rheumatol 11, 206-212, (2015).
Johnson, C. P., Tang, H. Y., Carag, C., Speicher, D. W. & Discher, D. E. Forced unfolding of proteins within cells. Science 317, 663-666, (2007).
Kerin, A. J., Wisnom, M. R. & Adams, M. A. The compressive strength of articular cartilage. Proc Inst Mech Eng H 212, 273-280, (1998).
Khoury L. R., N. J., Shmilovich K., Popa I. Study of Biomechanical Properties of Protein-Based Hydrogels Using Force-Clamp Rheometry. Macromolecules 51, 1441-1452, (2018).
Khoury, L. R. & Popa, I. Chemical unfolding of protein domains induces shape change in programmed protein hydrogels. Nat Commun 10, 5439, (2019).
Koga, N. et al. Principles for designing ideal protein structures. Nature 491, 222-227, (2012).
Kumru, B., Molinari, V., Dunnebacke, R., Blank, K. G. & Schmidt, B. Extremely Compressible Hydrogel via Incorporation of Modified Graphitic Carbon Nitride. Macromol Rapid Commun 40, e1800712, (2019).
Leahy, D. J., Hendrickson, W. A., Aukhil, I. & Erickson, H. P. Structure of a fibronectin type III domain from tenascin phased by MAD analysis of the selenomethionyl protein. Science 258, 987-991, (1992).
Leahy, D. J., Aukhil, I. & Erickson, H. P. 2.0 A crystal structure of a four-domain segment of human fibronectin encompassing the RGD loop and synergy region. Cell 84, 155-164, (1996).
Lei, H. et al. Single-Molecule Force Spectroscopy Trajectories of a Single Protein and Its Polyproteins Are Equivalent: A Direct Experimental Validation Based on A Small Protein NuG2. Angew Chem Int Ed Engl 56, 6117-6121, (2017).
Lei, H. et al. Stretchable hydrogels with low hysteresis and anti-fatigue fracture based on polyprotein cross-linkers. Nat Commun 11, 4032, (2020).
Li, H. et al. Reverse engineering of the giant muscle protein titin. Nature 418, 998-1002, (2002).
Li, Y., Xue, B., Cao, Y. Synthetic Protein Hydrogels. ACS Macro Lett 9, 512-524, (2020).
Li, L. et al. Biofabrication of a biomimetic supramolecular-polymer double network hydrogel for cartilage regeneration. Materials & Design 189, (2020).
Lillie, M. A., Chalmers, G. W. & Gosline, J. M. The effects of heating on the mechanical properties of arterial elastin. Connect Tissue Res 31, 23-35, (1994).
Linke, W. A., Popov, V. I. & Pollack, G. H. Passive and active tension in single cardiac myofibrils. Biophys J 67, 782-792, (1994).
Liu, J. et al. Tough Supramolecular Polymer Networks with Extreme Stretchability and Fast Room-Temperature Self-Healing. Adv Mater 29, (2017).
Lu, X. L. & Mow, V. C. Biomechanics of articular cartilage and determination of material properties. Med Sci Sports Exerc 40, 193-199, (2008).
Lv, S. et al. Designed biomaterials to mimic the mechanical properties of muscles. Nature 465, 69-73, (2010).
Mccutchen, C. W. Lubrication of Joints, The Joiints and Synovial Fluid. Vol. 10 437 (Academic, 1978).
McGann, C. L., Levenson, E. A. & Kiick, K. L. Resilin-Based Hybrid Hydrogels for Cardiovascular Tissue Engineering. Macromolecules 214, 203-213, (2013).
Minajeva, A., Kulke, M., Fernandez, J. M. & Linke, W. A. Unfolding of titin domains explains the viscoelastic behavior of skeletal myofibrils. Biophys J 80, 1442-1451, (2001).
Nakayama, A., Kakugo, A. Gong, J. P., Osada, Y., Takai M., Erata, M., Kawano S. High Mechanical Strength Double-Network Hydrogel with Bacterial Cellulose. Adv Mater 14, 1124-1128, (2004).
Numata, K., Ifuku, N., Masunaga, H., Hikima, T. & Sakai, T. Silk Resin with Hydrated Dual Chemical-Physical Cross-Links Achieves High Strength and Toughness. Biomacromolecules 18, 1937-1946, (2017).
O'Driscoll, S. W., Keeley, F. W. & Salter, R. B. Durability of regenerated articular cartilage produced by free autogenous periosteal grafts in major full-thickness defects in joint surfaces under the influence of continuous passive motion. A follow-up report at one year. J Bone Joint Surg Am 70, 595-606, (1988).
Okumura, K. I., K. The polyrotaxane gel: A topological gel by figure-of-eight cross-links. Adv Mater 13, 485-487, (2001).
Partlow, B. P. et al. Highly tunable elastomeric silk biomaterials. Adv Funct Mater 24, 4615-4624, (2014).
Partlow, B. P., Applegate, M. B., Omenetto, F. G. and Kaplan, D. L. Dityrosine Cross-Linking in Designing Biomaterials. ACS Biomater Sci Eng, 2108-2121, (2016).
Ren, X. et al. Super-tough, ultra-stretchable and strongly compressive hydrogels with core-shell latex particles inducing efficient aggregation of hydrophobic chains. Soft Matter 13, 3352-3358, (2017).
Rief, M., Gautel, M., Oesterhelt, F., Fernandez, J. M. & Gaub, H. E. Reversible unfolding of individual titin immunoglobulin domains by AFM. Science 276, 1109-1112, (1997).
Stanish, W. D. et al. Novel scaffold-based BST-CarGel treatment results in superior cartilage repair compared with microfracture in a randomized controlled trial. J Bone Joint Surg Am 95, 1640-1650, (2013).
Su, D. et al. Enhancing Mechanical Properties of Silk Fibroin Hydrogel through Restricting the Growth of beta-Sheet Domains. ACS Appl Mater Interfaces 9, 17489-17498, (2017).
Sun, J. Y. et al. Highly stretchable and tough hydrogels. Nature 489, 133-136, (2012).
Sun, W. et al. Molecular engineering of metal coordination interactions for strong, tough, and fast-recovery hydrogels. Sci Adv 6, eaaz9531, (2020).
Temple, D. K., Cederlund, A. A., Lawless, B. M., Aspden, R. M. & Espino, D. M. Viscoelastic properties of human and bovine articular cartilage: a comparison of frequency-dependent trends. BMC Musculoskelet Disord 17, 419, (2016).
Treloar, L. R. G. The physics of rubber elasticity. (Oxford University Press, USA, 1975).
van den Borne, M. P. et al. International Cartilage Repair Society (ICRS) and Oswestry macroscopic cartilage evaluation scores validated for use in Autologous Chondrocyte Implantation (ACI) and microfracture. Osteoarthritis Cartilage 15, 1397-1402, (2007).
Wainwright, S. A., Biggs, W. D., Currey, J. D., Gosline, J. M. Mechanical Design in Organisms. (Princeton University Press, 1982).
Wang, J., Lin, L., Cheng, Q. & Jiang, L. A strong bio-inspired layered PNIPAM-clay nanocomposite hydrogel. Angew Chem Int Ed Engl 51, 4676-4680, (2012).
Williamson, A. K., Chen, A. C., Masuda, K., Thonar, E. J. & Sah, R. L. Tensile mechanical properties of bovine articular cartilage: variations with growth and relationships to collagen network components. J Orthop Res 21, 872-880, (2003).
Wu, J. et al. Rationally designed synthetic protein hydrogels with predictable mechanical properties. Nat Commun 9, 620, (2018).
Xu, L., Zhao, X., Xu, C. & Kotov, N. A. Water-Rich Biomimetic Composites with Abiotic Self-Organizing Nanofiber Network. Adv Mater 30, (2018).
Xu, L. et al. Conjoined-network rendered stiff and tough hydrogels from biogenic molecules. Sci Adv 5, eaau3442, (2019).
Yokota, M., Yasuda, K., Kitamura, N., Arakaki, K., Onodera, S., Kurokawa., T., Gong, J. Spontaneous hyaline cartilage regeneration can be induced in an osteochondral defect created in the femoral condyle using a novel doublenetwork hydrogel. BMC Musculoskelet. Disord. 12, 49, (2011).
Zhao, X. Multi-scale multi-mechanism design of tough hydrogels: building dissipation into stretchy networks. Soft Matter 10, 672-687, (2014).

1. A protein hydrogel comprising a crosslinked network of entangled polypeptide chains, wherein the polypeptide chains comprise at least one amino acid sequence capable of functioning as a biochemical cue for tissue repair.

2. The protein hydrogel of claim 1, wherein the biochemical cue is for cartilage repair, bone repair or combinations thereof.

3. The protein hydrogel of claim 1 or 2, wherein the biochemical cue promotes stem cell adhesion, migration and/or differentiation.

4. The protein hydrogel of any one of claims 1 to 3, wherein the at least one amino acid sequence capable of functioning as a biochemical cue comprises a motif capable of entailing cell adhesion to a protein.

5. The protein hydrogel of any one of claims 1 to 4, wherein the polypeptide chains further comprise at least one folded globular domain.

6. The protein hydrogel of claim 5, wherein the at least one folded globular domain comprises ferredoxin-like folds.

7. The protein hydrogel of claim 5 or 6, wherein the protein hydrogel is derived from a protein comprising an unstructured protein sequence positioned between two folded globular domains.

8. The protein hydrogel of claim 7, wherein the unstructured protein sequence comprises one of the at least one amino acid sequences capable of functioning as a biochemical cue.

9. The protein hydrogel of any one of claims 1 to 8, wherein the protein hydrogel is derived from a protein comprising at least two different amino acid sequences capable of functioning as a biochemical cue, the first amino acid sequence having at least 90% sequence identity to the amino acid sequence set forth in SEQ ID NO:2 and the second amino acid sequence having at least 90% sequence identity to the amino acid sequence set forth in SEQ ID NO:3.

10. The protein hydrogel of claim 9, wherein the first amino acid sequence has the amino acid sequence set forth in SEQ ID NO:2 and the second amino acid sequence has the amino acid sequence set forth in SEQ ID NO:3.

11. The protein hydrogel of any one of claims 1 to 10, wherein the protein hydrogel is derived from a protein comprising an amino acid sequence having at least 90% sequence identity to the amino acid sequence set forth in SEQ ID NO:10.

12. The protein hydrogel of claim 11, wherein the protein hydrogel is derived from a protein comprising the amino acid sequence set forth in SEQ ID NO: 10.

13. The protein hydrogel of any one of claims 1 to 12, wherein the crosslinks comprise a disulfide bond between cysteine residues in the protein hydrogel.

14. The protein hydrogel of any one of claims 1 to 13, wherein the concentration of the crosslinked network of entangled polypeptide chains in the protein hydrogel is about 20% (w/v).

15. A method of preparing a protein hydrogel, the method comprising:
denaturing a protein in an aqueous environment to produce an aqueous composition comprising overlapping polypeptide chains;
crosslinking the polypeptide chains to produce a denatured protein hydrogel comprising a crosslinked network of entangled polypeptide chains; and
optionally at least partially renaturing the denatured protein hydrogel to produce a renatured protein hydrogel comprising a crosslinked network of entangled polypeptide chains,
wherein the protein comprises at least one amino acid sequence capable of functioning as a biochemical cue for tissue repair.

16. The method of claim 15, wherein the biochemical cue is for cartilage repair, bone repair or combinations thereof.

17. The method of claim 15 or 16, wherein the biochemical cue promotes stem cell adhesion, migration and/or differentiation.

18. The method of any one of claims 15 to 17, wherein the at least one amino acid sequence capable of functioning as a biochemical cue comprises a motif capable of entailing cell adhesion to a protein.

19. The method of any one of claims 15 to 18, wherein the protein further comprises at least one folded globular domain.

20. The method of claim 19, wherein the at least one folded globular domain comprises ferredoxin-like folds.

21. The method of claim 19 or 20, wherein the protein comprises an unstructured protein sequence positioned between two folded globular domains.

22. The method of claim 21, wherein the unstructured protein sequence comprises one of the at least one amino acid sequences capable of functioning as a biochemical cue.

23. The method of any one of claims 15 to 22, wherein the protein comprises at least two different amino acid sequences capable of functioning as biochemical cues, the first amino acid sequence having at least 90% sequence identity to the amino acid sequence set forth in SEQ ID NO:2 and the second amino acid sequence having at least 90% sequence identity to the amino acid sequence set forth in SEQ ID NO:3.

24. The method of claim 23, wherein the first amino acid sequence has the amino acid sequence set forth in SEQ ID NO:2 and the second amino acid sequence has the amino acid sequence set forth in SEQ ID NO:3.

25. The method of any one of claims 15 to 24, wherein the protein comprises the amino acid sequence set forth in SEQ ID NO:10 or comprises an amino acid sequence having at least 90% sequence identity to the amino acid sequence set forth in SEQ ID NO:10.

26. The method of any one of claims 15 to 25, wherein the denaturing comprises subjecting the protein to a chaotropic agent.

27. The method of claim 26, wherein the chaotropic agent comprises guanidinium chloride.

28. The method of any one of claims 15 to 27, wherein the concentration of the protein in the aqueous environment is about 20% (w/v).

29. The method of any one of claims 15 to 28, wherein the method comprises the renaturing of the denatured protein hydrogel to produce the renatured protein hydrogel.

30. The method of claim 29, wherein the renaturing comprises equilibrating the denatured protein hydrogel in phosphate buffered saline.

31. The method of any one of claims 15 to 30, wherein the crosslinking is carried out in a mold.

32. The method of any one of claims 15 to 31, wherein the crosslinks are prepared by a method compatible with use of the protein hydrogel in a subject.

33. The method of claim 32, wherein the crosslinks comprise a disulfide bond between cysteine residues obtained by a method comprising exposing the aqueous composition comprising the overlapping polypeptide chains to a source of oxygen.

34. A protein hydrogel prepared by a method as defined in any one of claims 15 to 33.

35. A use of a protein hydrogel of any one of claim 1 to 14 or 34 in repairing an osteochondral defect.

36. A synthetic protein comprising an amino acid sequence having at least 90% sequence identity to the amino acid sequence set forth in SEQ ID NO:10.

37. A synthetic protein having the amino acid sequence set forth in SEQ ID NO:10.

38. A use of the synthetic protein of claim 36 or 37 in preparing a protein hydrogel.

39. The use of claim 38, wherein the method is as defined in any one of claim 15 or 26-33.


SEQUENCE LISTING (11 sequences)

SEQ ID NO: 1 - protein, 82 aa, synthetic construct
MGEFDIRFRTDDDEQFEKVLKEMNRRARKDAGTVTYTRDGNDFEIRITGISEQNRKELAKEVERLAKEQNITVTYTERGSLE

SEQ ID NO: 2 - protein, 91 aa, synthetic construct
SRLDAPSQIEVKDVTDTTALITWFKPLAEIDGIELTYGIKDVPGDRTTIDLTEDENQYSIGNLKPDTEYEVSLISRRGDMSSNPAKETFTT

SEQ ID NO: 3 - protein, 17 aa, synthetic construct
TVYAVTGRGDSPASSRS

SEQ ID NO: 4 - protein, 684 aa, synthetic construct
MRGSHHHHHHGSMGEFDIRFRTDDDEQFEKVLKEMNRRARKDAGTVTYTRDGNDFEIRITGISEQNRKELAKEVERLAKEQNITVTYTERGSLERSMGEFDIRFRTDDDEQFEKVLKEMNRRARKDAGTVTYTRDGNDFEIRITGISEQNRKELAKEVERLAKEQNITVTYTERGSLERSMGEFDIRFRTDDDEQFEKVLKEMNRRARKDAGTVTYTRDGNDFEIRITGISEQNRKELAKEVERLAKEQNITVTYTERGSLERSMGEFDIRFRTDDDEQFEKVLKEMNRRARKDAGTVTYTRDGNDFEIRITGISEQNRKELAKEVERLAKEQNITVTYTERGSLERSMGEFDIRFRTDDDEQFEKVLKEMNRRARKDAGTVTYTRDGNDFEIRITGISEQNRKELAKEVERLAKEQNITVTYTERGSLERSMGEFDIRFRTDDDEQFEKVLKEMNRRARKDAGTVTYTRDGNDFEIRITGISEQNRKELAKEVERLAKEQNITVTYTERGSLERSMGEFDIRFRTDDDEQFEKVLKEMNRRARKDAGTVTYTRDGNDFEIRITGISEQNRKELAKEVERLAKEQNITVTYTERGSLERSMGEFDIRFRTDDDEQFEKVLKEMNRRARKDAGTVTYTRDGNDFEIRITGISEQNRKELAKEVERLAKEQNITVTYTERGSLERS

SEQ ID NO: 5 - protein, 684 aa, synthetic construct
MRGSHHHHHHGSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERS

SEQ ID NO: 6 - protein, 484 aa, synthetic construct
MRGSHHHHHHGSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERS

SEQ ID NO: 7 - protein, 484 aa, synthetic construct
MRGSHHHHHHGSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERS

SEQ ID NO: 8 - protein, 651 aa, synthetic construct
MRGSHHHHHHGSMDTYKLILNGKTLKGETTTEAVDAATAEKVFKQYANDNGVDGEWTYDDATKTFTVTERSGGRPSDSYGAPGGGNRSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSGGRPSDSYGAPGGGNRSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSMDTYKLILNGKTLKGETTTEAVDAATAEKVFKQYANDNGVDGEWTYDDATKTFTVTERSGGRPSDSYGAPGGGN

SEQ ID NO: 9 - protein, 592 aa, synthetic construct
MRGSHHHHHHGSMDTYKLVIVLNGTTFTYTTEAVDAATAEKVFKQYANDNGVDGEWTYDDATKTFTVTERSGGRPSDSYGAPGGGNRSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSGGRPSDSYGAPGGGNRSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSMDTYKLVIVLNGTTFTYTTEAVDAATAEKVFKQYANDNGVDGEWTYDDATKTFTVTERSGGRPSDSYGAPGGGN

SEQ ID NO: 10 - protein, 808 aa, synthetic construct
MRGSHHHHHHGSRLDAPSQIEVKDVTDTTALITWFKPLAEIDGIELTYGIKDVPGDRTTIDLTEDENQYSIGNLKPDTEYEVSLISRRGDMSSNPAKETFTTRSCMGEFDIRFRTDDDEQFEKVLKEMNRRARKDAGTVTYTRDGNDFEIRITGISEQNRKELAKEVERLAKEQNITVTYTERGSLRSCMGEFDIRFRTDDDEQFEKVLKEMNRRARKDAGTVTYTRDGNDFEIRITGISEQNRKELAKEVERLAKEQNITVTYTERGSLRSCMGEFDIRFRTDDDEQFEKVLKEMNRRARKDAGTVTYTRDGNDFEIRITGISEQNRKELAKEVERLAKEQNITVTYTERGSLRSCMGEFDIRFRTDDDEQFEKVLKEMNRRARKDAGTVTYTRDGNDFEIRITGISEQNRKELAKEVERLAKEQNITVTYTERGSLRSTVYAVTGRGDSPASSRSCMGEFDIRFRTDDDEQFEKVLKEMNRRARKDAGTVTYTRDGNDFEIRITGISEQNRKELAKEVERLAKEQNITVTYTERGSLRSCMGEFDIRFRTDDDEQFEKVLKEMNRRARKDAGTVTYTRDGNDFEIRITGISEQNRKELAKEVERLAKEQNITVTYTERGSLRSCMGEFDIRFRTDDDEQFEKVLKEMNRRARKDAGTVTYTRDGNDFEIRITGISEQNRKELAKEVERLAKEQNITVTYTERGSLRSCMGEFDIRFRTDDDEQFEKVLKEMNRRARKDAGTVTYTRDGNDFEIRITGISEQNRKELAKEVERLAKEQNITVTYTERGSLRSTVYAVTGRGDSPASS

SEQ ID NO: 11 - protein, 775 aa, synthetic construct
MRGSHHHHHHGSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSMGEFDIRFRTDDDEQFEKVLKECNRRARKDAGTVTYTRDGNDFEIRITGISEQNRKELAKEVERLAKEQNITVTYTERGSLERSSRLDAPSQIEVKDVTDTTALITWFKPLAEIDGIELTYGIKDVPGDRTTIDLTEDENQYSIGNLKPDTEYEVSLISRRGDMSSNPAKETFTT